(19) United States
(12) Patent Application Publication
(10) Pub. No.: US 2011/0301200 A1
(43) Pub. Date: Dec. 8, 2011

(54) THYROID RECEPTOR LIGANDS

(75) Inventors: Saurin Raval, Gujarat (IN); Preeti Raval, Gujarat (IN)

(73) Assignee: CADILA HEALTHCARE LIMITED, Ahmedabad, Gujarat (IN)

(21) Appl. No.: 13/126,167

(22) PCT Filed: Oct. 22, 2009
(86) PCT No.: PCT/IN2009/000598

§ 371 Date: Jun. 29, 2011

(30) Foreign Application Priority Data
Oct. 27, 2008 (IN) 2312/MUM/2008

(51) Int. Cl. A61K 31/4453; C07D 295/26; A61P 3/04; A61K 31/40; C07C 317/46; A61P 3/06; C07C 251/60; A61K 31/197

(52) U.S. Cl. 514/331; 562/440; 546/216; 562/430; 562/427; 548/542; 514/567; 514/562; 514/424; 560/35; 560/12

(57) ABSTRACT

The present invention relates to novel compounds of general formula (I) which are thyroid receptor ligands and are preferably selective for the thyroid hormone receptor beta (TR-Beta). Further, the present invention relates to processes of preparing such compounds, their tautomeric forms, novel intermediates involved in their synthesis, their pharmaceutically acceptable salts, methods for using such compounds and pharmaceutical compositions containing them.

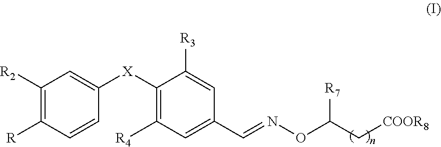

### FIELD OF THE INVENTION

The present invention relates to novel compounds of general formula (I) which are thyroid receptor (TR) ligands and are preferably selective for the thyroid hormone receptor beta including their tautomeric forms, isomers including their stereo & regioisomers, their pharmaceutically acceptable salts, their polymorphic forms as well as novel intermediates involved in their synthesis. Further, the present invention relates to processes of preparing such compounds, their tautomeric forms, novel intermediates involved in their synthesis, their pharmaceutically acceptable salts, methods for using such compounds and pharmaceutical compositions containing them.

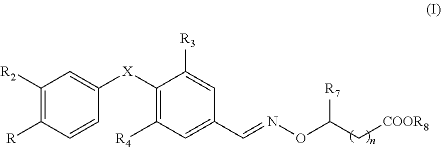

### BACKGROUND TO THE INVENTION

Thyroid hormones (TH) are synthesized in the thyroid in response to thyroid stimulating hormone (TSH), which is secreted by the pituitary gland. Production of T4, and T3, by the thyroid gland is under negative feedback control. TSH, also known as thyrotropin, is responsible for normal thyroid gland function and thyroid hormone secretion. It is synthesized in the anterior pituitary gland, and its secretion is controlled by thyroid releasing hormone (TRH) that is synthesized in the hypothalamus.

The natural thyroid hormones (TH) T₃ and T₄ is an important endocrine signaling hormone. Thyroid hormones are iodinated tyrosine analogues excreted into the circulation primarily as T4. T4 is converted to T3 rapidly by deiodination in local tissues which is the most potent thyroid hormone. It plays important role in normal development, differentiation and maintenance of metabolic balance, control of cholesterol levels through interaction with thyroid hormone receptors (THR). Natural thyroid hormone, T₃ exhibit its physiological effect by acting on a Thyroid Hormone Receptor (THR), which belongs to the nuclear hormone receptor super family. There are two different isoforms of Thyroid Hormone Receptors, THR-α and THR-β. Further, these two isoforms are sub-classified as α₁; α₂ and β₁; β₂ subtypes. THRβ₁ is prevalent in liver (85%), while THR α₁ is mainly present in cardiac tissue (Yen P. M. Physiol. Rev; 2001; 81:1097-1142).

At normal levels, T₃ maintains body weight, metabolic rate, body temperature, mood and regulate serum cholesterol. Hypothyroidism is associated with weight gain, high levels of low-density lipoproteins (LDL) cholesterol and depression. Hyperthyroidism leads to weight loss, hypermetabolism, lowering of serum LDL levels, cardiac arrhythmia, heart failure, muscle weakness, bone loss and anxiety.

The natural thyroid hormone T₃ does not show any selectivity in binding to both of the THR isoforms (THR α₁ and THR β₁). Thus, administration of T₃ lowers plasma cholesterol, low-density lipoprotein (LDL) and triglyceride levels in animal models and humans. However, T₃ cannot be used therapeutically to treat hypercholesterolemia and obesity due to its cardiac side effects such as tachycardia and arrhythmia. However, knockout animal studies as well as results with some selective ligands suggest that such cardiac side effects can be attributed to the THR α₁ isoform. Thus, some effects of T₃ may be therapeutically useful in non-thyroid disorders if adverse effects can be minimized or eliminated. These potentially useful influences include weight reduction, lowering of serum LDL levels, amelioration of depression and stimulation of bone formation (Cheng S. Steroids; 2005; 70: 450-454).

Development of specific and selective thyroid hormone receptor ligands, particularly THR β agonist could lead to specific therapies for disorders such as obesity and hyperlipidemia, while avoiding the cardiovascular and other toxicities of native thyroid hormones. Thus, compounds mimicking only the beneficial effects of the thyroid hormone and lacking their cardiac side effects (tachycardia and arrhythmia) potentially could be used to treat a number of conditions such as obesity and dyslipidemia. In this regard, THR agonists that interact selectively with the β isoform of the THR offer an especially attractive method for avoiding cardiotoxicity (J. D. Baxter. Trends Endocrinol. Metab. 2004;15:154-157). Selective THR β agonist exhibit modest cardiac sparing in rodents and primates and lower lipids but it may induce the THR β mediated suppression of the THA.

Two strategies have been attempted for the development of Thyromimetics.

One is by making isoform selective compounds (Johan Malm, J. Med. Chem. 2003, 46, 1580-1588) and another is by making Liver selective Thyromimetics (Mark D. Erion, PNAS 2007 15490-15495). Liver selective compounds are expected not to suppress the thyroid hormone axis (THA). Thus thyromimetic which has isoform selectivity incorporated with liver selectivity can be expected to be devoid of cardiac toxicity and will not suppress THA.

Various compounds have been disclosed as possible agonists of THR including those which claim to be liver selective. Some of the more relevant ones for the present invention includes WO 0039077, WO 2004067482, U.S. Pat. No. 6,344,481, U.S. Pat. No. 6,787,652, US20070173548, WO2006128058, WO 20080221210 and WO 2009089093 which are incorporated herein as reference.

However, none of these compounds have been commercially developed and looking at the beneficial potential and medical need for such compounds, specifically compounds having better liver selectivity while retaining its therapeutic efficacy, there remains a need for developing further compounds with better therapeutic and/or safety profile. Herein, we disclose novel compounds which shows activity as THR β agonists, some of which also have better liver selectivity.

### SUMMARY OF THE INVENTION

The present invention describes novel compounds that are thyroid receptor (TR) ligands and are preferably selective for the thyroid hormone receptor beta 1, which are useful for the treatment of a number of conditions such as obesity and dyslipidemia. The novel compounds are defined by the general formula (I) as given below.

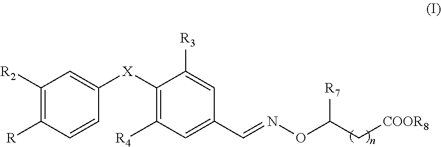

The compounds of the present invention are useful in the treatment of the human or animal body, by regulation of selective thyroid hormone receptor gene expression. The compounds of this invention are therefore suitable for the treatment/mitigation/regulation or prophylaxis of obesity and dyslipidemia.

### Preferred Embodiments

The main objective of the present invention is to provide novel compounds of general formula (I), their tautomeric forms, novel intermediates involved in their synthesis, their pharmaceutically acceptable salts, their pharmaceutically acceptable solvates and pharmaceutical compositions containing them or their mixtures suitable for the treatment of obesity and dyslipidemia.

In an embodiment is provided a process for the preparation of novel compounds of general formula (I), their tautomeric forms, isomers including their stereo & regioisomers, novel intermediates involved in their synthesis, their pharmaceutically acceptable salts, pharmaceutically acceptable solvates, polymorphic forms and pharmaceutical compositions containing them.

In another embodiment is provided pharmaceutical compositions containing compounds of general formula (I), their tautomeric forms, their pharmaceutically acceptable salts, solvates and their mixtures having pharmaceutically acceptable carriers, solvents, diluents, excipients and other media normally employed in their manufacture.

In a further another embodiment is provided the use of the novel compounds of the present invention for the treatment of obesity and dyslipidemia, by administering a therapeutically effective & non-toxic amount of the compound of formula (I), or their pharmaceutically acceptable compositions to the mammals.

### DETAILED DESCRIPTION

Accordingly, the present invention relates to compounds of the general formula (I),

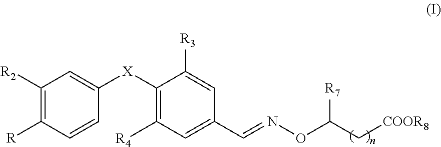

wherein R═OR₁, NHR₁
wherein R₁ may be selected from H, optionally substituted groups selected from linear or branched (C₁-C₆)alkyl, (C₃-C₇)cycloalkyl, acyl, aryl, aralkyl groups;
R₂ represents hydrogen, hydroxyl, halo, optionally substituted groups selected from (C₁-C₆)alkyl, (C₃-C₇)cycloalkyl, aryl, heteroaryl, acyl, oxo, aryloxy, aralkyl, aralkoxy, carboxylic acid and its derivatives such as esters and amides, sulfenyl derivatives, sulfonyl derivatives, sulfonic acid and its derivatives; or the groups —CONR₅R₆, —SO₂NR₅R₆, wherein
R₅ & R₆ may be same or different and are independently selected from H, optionally substituted groups selected from linear or branched (C₁-C₆)alkyl, (C₃-C₇)cycloalkyl, bicycloalkyl, aryl or the groups R₅ & R₆ together with the nitrogen atom to which they are attached, form a five to eight membered cyclic ring which may further optionally contain one or more heteroatoms selected from N, S, O;
R₃, R,₄ may be independently selected from H, halogen, (C₁-C₆)alkyl groups; X is selected from O, —CH₂—, CO;
R₇ may be selected from H, optionally substituted groups selected from linear or branched (C₁-C₆)alkyl, (C₃-C₇)cycloalkyl, acyl, aryl, aralkyl, heteroaryl groups and ‘n’ represents integers from 0-2;
R₈ may be selected from H, optionally substituted groups selected from linear or branched (C₁-C₆)alkyl groups;
   In a preferred embodiment,
R₂ is selected linear or branched (C₁-C₆)alkyl, phenyl, benzoyl benzyl, carboxamide and sulfonamide groups, each of these groups being further substituted with suitable substituents and R₈ represents an (C₁-C₆)alkyl group.

In a still preferred embodiment, the aryl group is selected from phenyl, naphthyl, tetrahydronaphthyl, indane, biphenyl groups; the heteroaryl group is selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl group;

The substituents on alkyl, aryl, aralkyl, aryloxy, aralkoxy, heteroaryl or cycloalkyl groups as defined above may be selected from hydroxyl, halo, cyano, optionally substituted groups selected from (C₁-C₆)alkyl, haloalkyl; alkoxy, oxo, aryl, aryloxy, aralkyl, acyl, alkylthio, thioalkyl groups, with the further option that when any of these groups are further substituted, the substituents on these substitutes may be selected from any of the groups described above;

In a still preferred embodiment, the substitutions on R₂ when present, is selected from halogen, hydroxy, amino, alkyl, haloalkyl, alkoxy groups.

In a preferred embodiment, the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, iso-hexyl and the like;
the “cycloalkyl” or “alicyclic” group used either alone or in combination with other radicals, is selected from a cyclic radical containing three to seven carbons, more preferably cyclopropyl, cyclobutyl, cyclopentyl, cyclohexyl, cycloheptyl and the like;
the “alkoxy” group used either alone or in combination with other radicals, is selected from groups containing an alkyl radical, as defined above, attached directly to an oxygen atom, more preferably groups selected from methoxy, ethoxy, n-propoxy, iso-propoxy, n-butoxy, t-butoxy, iso-butoxy, pentyloxy, hexyloxy, and the like;
the “haloalkyl” group is selected from an alkyl radical, as defined above, suitably substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “aryl” or “aromatic” group used either alone or in combination with other radicals, is selected from a suitable aromatic system containing one, two or three rings wherein such rings may be attached together in a pendant manner or may be fused, more preferably the groups are selected from phenyl, naphthyl, tetrahydronaphthyl, indane, biphenyl, and the like;
the “aralkyl” group used above either alone or in combination represents an alkyl group as defined above attached to an aryl group;
“arylkoxy” group used above either alone or in combination represents an alkoxy group as defined above attached to an aryl group;
“aryloxy” group used above either alone or in combination represents an oxygen atom linked to an aryl group;
the “heteroaryl” or “heteroaromatic” group used either alone or in combination with other radicals, is selected from suitable single or fused mono, bi or tricyclic aromatic heterocyclic radicals containing one or more hetero atoms selected from O, N or S, more preferably the groups are selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl and the like;
the “acyl” group used either alone or in combination with other radicals, is selected from a radical containing one to eight carbons, more preferably selected from formyl, acetyl, propanoyl, butanoyl, iso-butanoyl, pentanoyl, hexanoyl, heptanoyl, benzoyl and the like, which may be substituted;
the “oxo” or “carbonyl” group used either alone (—C═O—) or in combination with other radicals such as alkyl described above, for e.g. “alkylcarbonyl”, denotes a carbonyl radical (—C═O—) substituted with an alkyl radical described above such as acyl or alkanoyl;
the “carboxylic acid” group, used alone or in combination with other radicals, denotes a —COOH group, and includes derivatives of carboxylic acid such as esters and amides;
the “ester” group used alone or in combination with other radicals, denotes —COO— group, and includes carboxylic acid derivatives, more preferably the ester moieties are selected from alkoxycarbonyl, such as methoxycarbonyl, ethoxycarbonyl, and the like, which may optionally be substituted; aryloxycarbonyl group such as phenoxycarbonyl, napthyloxycarbonyl, and the like, which may optionally be substituted; aralkoxycarbonyl group such as benzyloxycarbonyl, phenethyloxycarbonyl, napthylmethoxycarbonyl, and the like, which may optionally be substituted; heteroaryloxycarbonyl, heteroaralkoxycarbonyl, wherein the heteroaryl group, is as defined above, which may optionally be substituted; heterocyclyloxycarbonyl, where the heterocyclic group, as defined earlier, which may optionally be substituted;
the “amide” group used alone or in combination with other radicals, represents an aminocarbonyl radical (H₂N—C═O—), wherein the amino group is mono- or di-substituted or unsubstituted, more preferably the groups are selected from methylamide, dimethylamide, ethylamide, diethylamide, and the like;
the “alkylthio” group used either alone or in combination with other radicals, denotes a straight or branched or cyclic monovalent substituent comprising an alkyl group as defined above, linked through a divalent sulfur atom having a free valence bond from the sulfur atom, more preferably the groups may be selected from methylthio, ethylthio, propylthio, butylthio, pentylthio and the like or cyclic alkylthio selected from cyclopropylthio, cyclobutylthio, cyclopentylthio, cyclohexylthio and the like, which may be optionally substituted;
the “thioalkyl” group used either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be optionally substituted;
the “sulfenyl” group or “sulfenyl derivatives” used alone or in combination with other radicals, represents a bivalent group, —SO— or RₓSO, where Rₓ is an optionally substituted alkyl, aryl, heteroaryl, heterocyclyl, group selected from those described above;
the “sulfonyl” group or “sulfones derivatives” used either alone or in combination with other radicals, with other terms such as alkylsulfonyl, represents a divalent radical —SO₂—, or RₓSO₂—, where Rₓ is as defined above. More preferably, the groups may be selected from “alkylsulfonyl” wherein suitable alkyl radicals, selected from those defined above, is attached to a sulfonyl radical, such as methylsulfonyl, ethylsulfonyl, propylsulfonyl and the like, “arylsulfonyl” wherein an aryl radical, as defined above, is attached to a sulfonyl radical, such as phenylsulfonyl and the like.

Suitable groups and substituents on the groups may be selected from those described anywhere in the specification.

Preferred compounds according to the present invention include but not limited to:
2-(((3,5-dichloro-4-(4-hydroxy-3-isopropyl phenoxy)benzylidene)amino)oxy)acetic acid;
2-(((4-(3-(sec-butyl)-4-hydroxyphenoxy)-3,5-dichlorobenzylidene)amino)oxy)acetic acid;
2-(((3,5-dibromo-4-(4-hydroxy-3-isopropylphenoxy)benzylidene)amino)oxy)acetic acid;
2-(((3,5-dichloro-4-((6-hydroxy-[1,1′-biphenyl]-3-yl)oxy)benzylidene)amino)oxy)acetic acid;
2-(((4-(3-benzyl-4-hydroxyphenoxy)-3,5-dichlorobenzylidene)amino)oxy)acetic acid;
2-(((3,5-dibromo-4-(3-(sec-butyl)-4-hydroxyphenoxy)benzylidene)amino)oxy)acetic acid;
2-(((3,5-dibromo-4-((6-hydroxy-[1,1′-biphenyl]-3-yl)oxy)benzylidene)amino)oxy)acetic acid;
2-(((3,5-dichloro-4-(4-hydroxy-3-isopropylphenoxy)benzylidene)amino)oxy)propanoic acid;
2-4(4-(3-benzyl-4-hydroxyphenoxy)-3,5-dibromobenzylidene)amino)oxy)acetic acid;
2-(((3,5-dibromo-4-(4-hydroxy-3-isopropylphenoxy)benzylidene)amino)oxy)propanoic acid;
2-(((3,5-dibromo-4-((6-hydroxy-[1,1′-biphenyl]-3-yl)oxy)benzylidene)amino)oxy)propanoic acid;
2-(((3,5-dibromo-4-(4-hydroxy-3-isopropylphenoxy)benzylidene)amino)oxy)-2-methylpropanoic acid;
2-(((3,5-dibromo-4-(4-hydroxy-3-isopropylphenoxy)benzylidene)amino)oxy)butanoic acid;
2-(((3,5-dibromo-4-(3-(sec-butyl)-4-hydroxyphenoxy)benzylidene)amino)oxy)propanoic acid;
2-(((3,5-dibromo-4-(3-(sec-butyl)-4-hydroxyphenoxy)benzylidene)amino)oxy)-2-methyl prop anoic acid;
2-(((4-(3-benzyl-4-hydroxyphenoxy)-3,5-dibromobenzylidene)amino)oxy)propanoic acid;
2-(((4-(3-benzyl-4-hydroxyphenoxy)-3,5-dibromobenzylidene)amino)oxy)butanoic acid;
2-(((3,5-dibromo-4-(3-(sec-butyl)-4-hydroxyphenoxy)benzylidene)amino)oxy)-2-phenylacetic acid;
2-(((3,5-dibromo-4-(3-(sec-butyl)-4-hydroxyphenoxy)benzylidene)amino)oxy)butanoic acid;
2-(((3,5-dibromo-4-((6-hydroxy-[1,1′-biphenyl]-3-yl)oxy)benzylidene)amino)oxy)-2-methyl propanoic acid;
2-(((3,5-dibromo-4-((6-hydroxy-[1,1′-biphenyl]-3-yl)oxy)benzylidene)amino)oxy)butanoic acid;
2-(((4-(3-benzyl-4-hydroxyphenoxy)-3,5-dibromobenzylidene)amino)oxy)-2-methyl propanoic acid;
2-(((4-(3-(sec-butyl)-4-hydroxyphenoxy)-3,5-dichlorobenzylidene)amino)oxy)propanoic acid;
2-(((3,5-dibromo-4-(3-(tert-butyl)-4-hydroxyphenoxy)benzylidene)amino)oxy)propanoic acid;
2-(((3,5-dibromo-4-(3-ethyl-4-hydroxyphenoxy)benzylidene)amino)oxy)propanoic acid;
2-(((3,5-dibromo-4-(3-(4-chlorobenzoyl)-4-hydroxy phenoxy)benzylidene)amino)oxy)propan oic acid;
2-(((3,5-dibromo-4-(3-((4-chlorophenyl)(hydroxy)methyl)-4-hydroxy phenoxy)benzylidene)amino)oxy)propanoic acid;
2-(((3,5-dibromo-4-(3-(4-chlorobenzoyl)-4-hydroxyphenoxy)benzylidene)amino)oxy)acetic acid;
2-(((3,5-dibromo-4-(3-((4-chlorophenyl)(hydroxy)methyl)-4-hydroxyphenoxy)benzylidene)amino)oxy)acetic acid;
2-(((3,5-dibromo-4-(3-(3-chlorobenzoyl)-4-hydroxy phenoxy)benzylidene)amino)oxy)propanoic acid;
2-(((3,5-dibromo-4-(3-(4-bromobenzoyl)-4-hydroxyphenoxy)benzylidene)amino)oxy)acetic acid;
2-(((3,5-dibromo-4-(3-((4-bromophenyl)(hydroxy)methyl)-4-hydroxyphenoxy)benzylidene)amino)oxy)acetic acid;
2-(((3,5-dibromo-4-(3-((3-chlorobenzoyl)-4-hydroxyphenoxy)benzylidene)amino)oxy)acetic acid;
2-(((3,5-dibromo-4-(3-((3-chlorophenyl)(hydroxy)methyl)-4-hydroxyphenoxy)benzylidene)amino)oxy)propanoic acid;
2-(((3,5-dibromo-4-(3-((3-chlorophenyl)(hydroxy)methyl)-4-hydroxyphenoxy)benzylidene)amino)oxy)acetic acid;
2-(((3,5-dibromo-4-(4-hydroxy-3-(piperidin-1-ylsulfonyl)phenoxy)benzylidene)amino)oxy)propanoic acid;
2-(((3,5-dibromo-4-(4-hydroxy-3-(N-isopropylsulfamoyl)phenoxy)benzylidene)amino)oxy)pro panoic acid;
2-(((3,5-dibromo-4-(3-(N,N-diethylsulfamoyl)-4-hydroxyphenoxy)benzylidene)amino)oxy)pro panoic acid;
2-(((3,5-dibromo-4-(3-(N-cyclohexylsulfamoyl)-4-hydroxyphenoxy)benzylidene)amino)oxy)propanoic acid;
2-(((4-(3-(N-((1R,2R,4S)-bicyclo[2.2.1]heptan-2-yl)sulfamoyl)-4-hydroxyphenoxy)-3,5-dibromobenzylidene)amino)oxy)propanoic acid;
2-(((3,5-dibromo-4-(4-hydroxy-3-(pyrrolidin-1-ylsulfonyl)phenoxy)benzylidene)amino)oxy)acetic acid;
Ethyl 2-(((3,5-dichloro-4-(3-isopropyl-4-methoxyphenoxy)benzylidene)amino)oxy)acetate;
Ethyl 2-(((4-(3-(sec-butyl)-4-methoxyphenoxy)-3,5-dichlorobenzylidene)amino)oxy)acetate;
Ethyl 2-(((3,5-dibromo-4-(3-isopropyl-4-methoxyphenoxy)benzylidene)amino)oxy)acetate;
Ethyl 2-(((3,5-dichloro-4-((6-methoxy-[1,1′-biphenyl]-3-yl)oxy)benzylidene)amino)oxy)acetate;
Ethyl 2-(((4-(3-benzyl-4-methoxyphenoxy)-3,5-dichlorobenzylidene)amino)oxy)acetate;
Ethyl 2-(((3,5-dibromo-4-(3-(sec-butyl)-4-methoxyphenoxy)benzylidene)amino)oxy)acetate;
Ethyl 2-(((3,5-dibromo-4-((6-methoxy-[1,1′-biphenyl]-3-yl)oxy)benzylidene)amino)oxy)acetate;
Ethyl 2-(((3,5-dichloro-4-(3-isopropyl-4-methoxyphenoxy)benzylidene)amino)oxy)propanoate;
Ethyl 2-(((4-(3-benzyl-4-methoxyphenoxy)-3,5-dibromobenzylidene)amino)oxy)acetate;
Ethyl 2-(((3,5-dibromo-4-(3-isopropyl-4-methoxyphenoxy)benzylidene)amino)oxy)propanoate;
Ethyl 2-(((3,5-dibromo-4-((6-methoxy-[1,1′-biphenyl]-3-yl)oxy)benzylidene)amino)oxy)propanoate;
Ethyl 2-(((3,5-dibromo-4-(3-isopropyl-4-methoxyphenoxy)benzylidene)amino)oxy)-2-methyl propanoate;
Ethyl 2-(((3,5-dibromo-4-(3-isopropyl-4-methoxyphenoxy)benzylidene)amino)oxy)butanoate;
Ethyl 2-(((3,5-dibromo-4-(3-(sec-butyl)-4-methoxyphenoxy)benzylidene)amino)oxy)propanoate;
Ethyl 2-(((3,5-dibromo-4-(3-(sec-butyl)-4-methoxyphenoxy)benzylidene)amino)oxy)-2-methylpropanoate;
Ethyl 2-(((4-(3-benzyl-4-methoxyphenoxy)-3,5-dibromobenzylidene)amino)oxy)propanoate;
Ethyl 2-(((4-(3-benzyl-4-methoxyphenoxy)-3,5-dibromobenzylidene)amino)oxy)butanoate;
Ethyl 2-(((3,5-dibromo-4-(3-(sec-butyl)-4-methoxyphenoxy)benzylidene)amino)oxy)-2-phenyl acetate;
Ethyl 2-(((3,5-dibromo-4-(3-(sec-butyl)-4-ethoxy phenoxy)benzylidene)amino)oxy)butanoate;
Ethyl 2-(((3,5-dibromo-4-((6-methoxy-[1,1′-biphenyl]-3-yl)oxy)benzylidene)amino)oxy)-2-methylpropanoate;
Ethyl 2-(((3,5-dibromo-4-((6-methoxy-[1,1′-biphenyl]-3-yl)oxy)benzylidene)amino)oxy)butanoate;
Ethyl 2-(((4-(3-benzyl-4-methoxyphenoxy)-3,5-dibromobenzylidene)amino)oxy)-2-methyl propanoate;
Ethyl 2-(((4-(3-(sec-butyl)-4-methoxyphenoxy)-3,5-dichlorobenzylidene)amino)oxy)propanoate;
Ethyl 2-(((3,5-dibromo-4-(3-(tert-butyl)-4-methoxyphenoxy)benzylidene)amino)oxy)propanoate;
Ethyl 2-(((3,5-dibromo-4-(3-ethyl-4-methoxyphenoxy)benzylidene)amino)oxy)propanoate;
Ethyl 2-(((3,5-dibromo-4-(3-(4-chlorobenzoyl)-4-methoxyphenoxy)benzylidene)amino)oxy)propanoate;
Ethyl 2-(((3,5-dibromo-4-(3-(4-chlorobenzoyl)-4-hydroxyphenoxy)benzylidene)amino)oxy)propanoate;
Ethyl 2-(((3,5-dibromo-4-(3-(4-chlorobenzoyl)-4-hydroxyphenoxy)benzylidene)amino)oxy)acetate;
Ethyl 2-(((3,5-dibromo-4-(3-(3-chlorobenzoyl)-4-hydroxyphenoxy)benzylidene)amino)oxy)propanoate;
Ethyl 2-(((3,5-dibromo-4-(3-(4-bromobenzoyl)-4-hydroxyphenoxy)benzylidene)amino)oxy)acetate;
Ethyl 2-(((3,5-dibromo-4-(3-(3-chlorobenzoyl)-4-hydroxyphenoxy)benzylidene)amino)oxy)acetate;
Ethyl 2-(((3,5-dibromo-4-(4-methoxy-3-(piperidin-1-ylsulfonyl)phenoxy)benzylidene)amino)oxy)propanoate;
Ethyl 2-(((3,5-dibromo-4-(3-(N-isopropylsulfamoyl)-4-methoxyphenoxy)benzylidene)amino)oxy)propanoate;
Ethyl 2-(((3,5-dibromo-4-(3-(N,N-diethylsulfamoyl)-4-methoxyphenoxy)benzylidene)amino)oxy)propanoate;
Ethyl 2-(((3,5-dibromo-4-(3-(N-cyclohexylsulfamoyl)-4-methoxyphenoxy)benzylidene)amino)oxy)propanoate;
Ethyl 2-(((4-(3-(N-((1R,2R,4S)-bicyclo[2.2.1]heptan-2-yl)sulfamoyl)-4-methoxyphenoxy)-3,5-dibromobenzylidene)amino)oxy)propanoate;
Ethyl 2-(((3,5-dibromo-4-(4-methoxy-3-(pyrrolidin-1-ylsulfonyl)phenoxy)benzylidene)amino)oxy)acetate;
Ethyl 2-(((3,5-dibromo-4-(4-methoxy-3-(pyrrolidin-1-ylsulfonyl)phenoxy)benzylidene)amino)oxy)propanoate;

The compounds of this invention may be prepared using the reactions and techniques described in the following section including the schemes 1-4. The reactions are performed in solvents appropriate to the reagents and materials employed and are suitable for the transformations being effected. It is understood by those skilled in the art that the nature and order of the synthetic steps presented may be varied for the purpose of optimizing the formation of the compounds of the present invention. It will also be appreciated that some routine alterations/modifications including requirement of one or more additional steps which may be required for obtaining the compounds of the present invention in preferred yields but are considered to be within the scope of a person skilled in the art, are to be considered to be within the scope of the present invention.

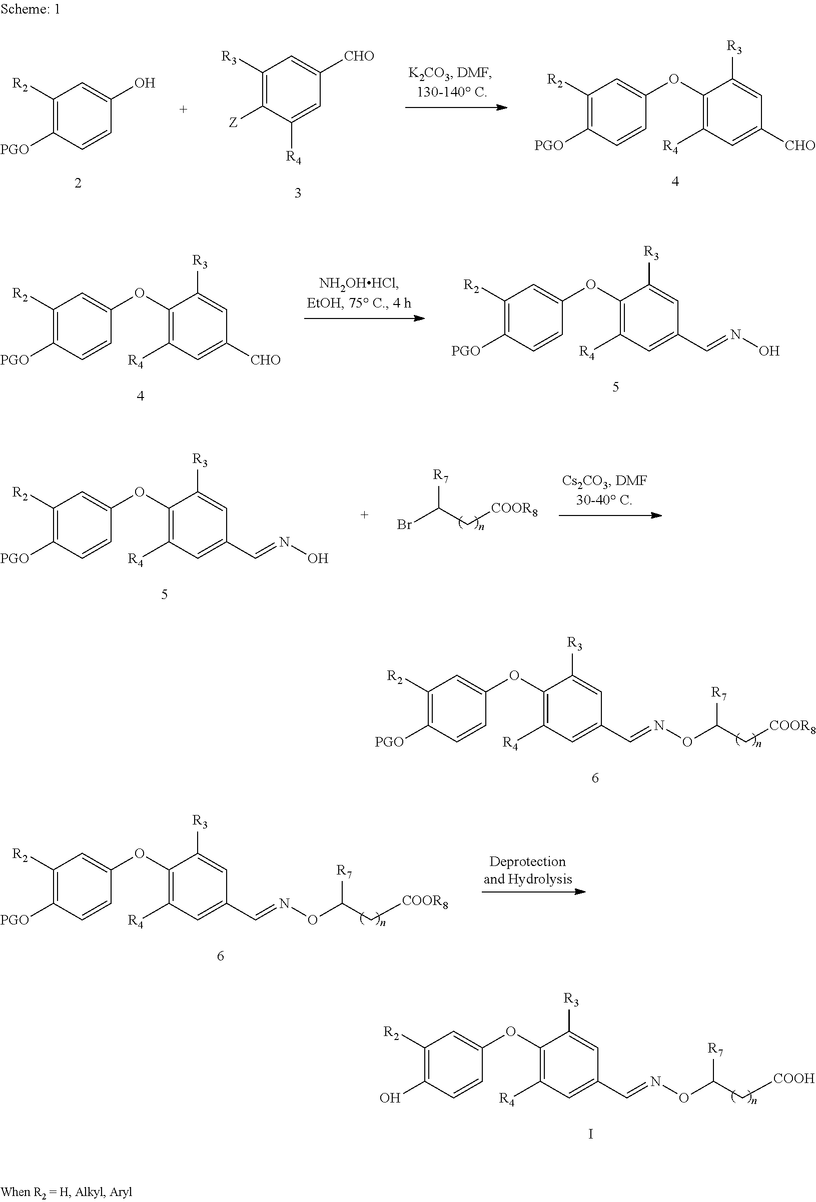

Reacting protected phenol of formula 2, wherein PG represents suitable protecting groups known to persons skilled in the art (for e.g. those described in T. W. Greene and P. G. M. Wuts “Protective groups in Organic Synthesis”, John Wiley & Sons, Inc, 1999, 3^{rd} Ed., 201-245 along with references therein), & R₂ is as defined earlier, with aldehyde compound of formula 3 wherein R₃ and R₄ are as defined earlier and ‘Z’ is halogen, to give coupled product 4. The reaction may be carried out in solvents such as DMF, DMSO, THF, toluene and the like or their suitable mixtures, in the presence of a base such as K₂CO₃, NaH, KOH and the like or their suitable mixtures. Reaction of coupled product 4 with hydroxyl amine hydrochloride afforded oxime compound of formula 5. Alkylation of the oxime compound of formula 5 with bromo alkyl esters (where R₇ & R₈ are as defined earlier) using suitable base such as K₂CO₃, Cs₂CO₃, KOH, NaH and the like afforded ester compound of formula 6. Deprotection and hydrolysis of compound 6 using appropriate reagents will lead to compound of formula (I).

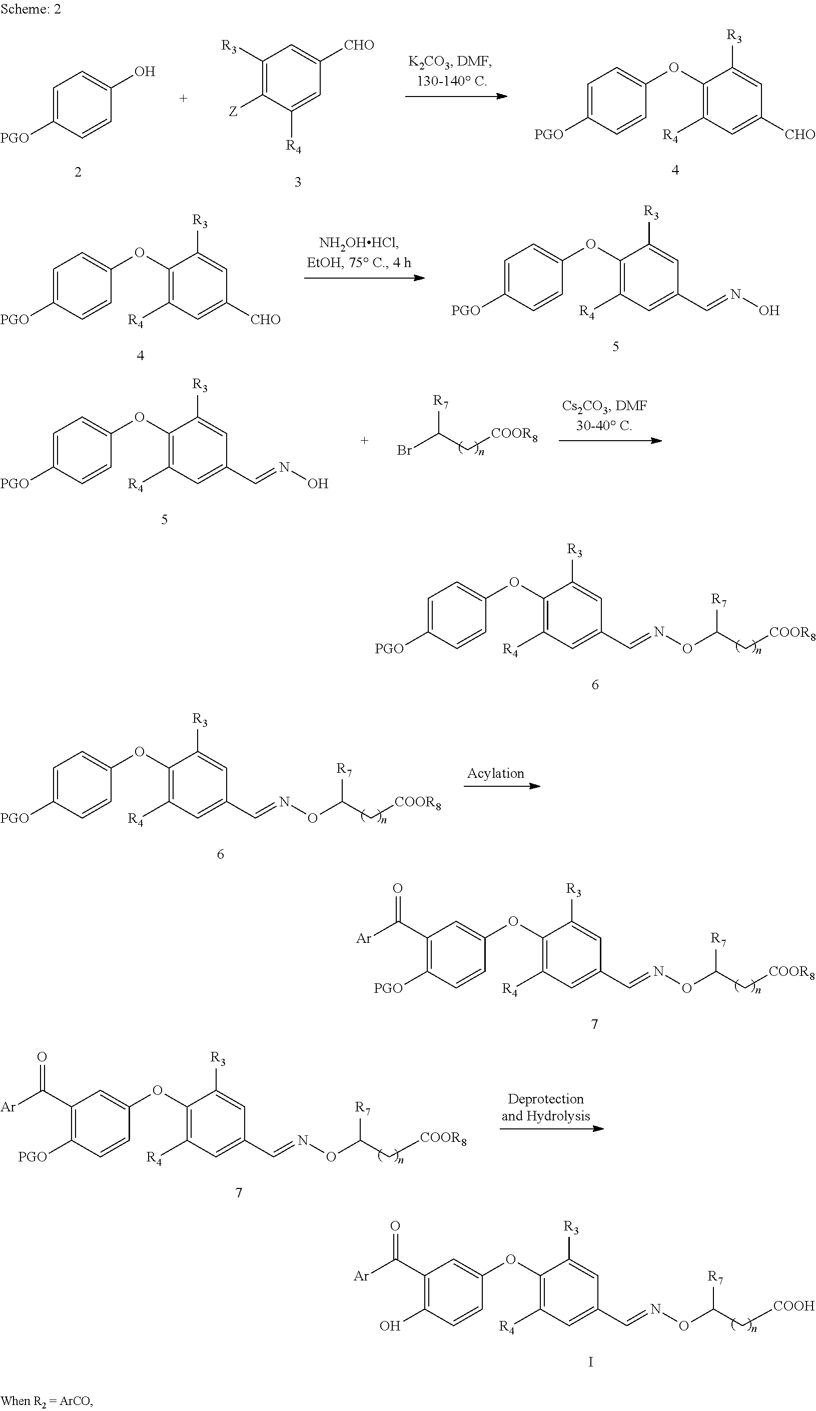

Reacting protected phenol of formula 2, wherein ‘PG’ represents suitable protecting groups known to persons skilled in the art (for e.g. those described in T. W. Greene and P. G. M. Wuts “Protective groups in Organic Synthesis”, John Wiley &

Sons, Inc, 1999, 3^{rd} Ed., 201-245 along with references therein), & R₂ is as defined earlier, with aldehyde compound of formula 3 wherein R₃ and R₄ are as defined earlier and ‘Z’ is halogen to give coupled product 4. The reaction may be carried out in solvents such as DMF, DMSO, THF, toluene and the like or their suitable mixtures, in the presence of a base such as K₂CO₃, NaH, KOH and the like or their suitable mixtures. Reaction of coupled product 4 with hydroxyl amine hydrochloride afforded oxime compound of formula 5. Alkylation of the oxime compound of formula 5 with bromo alkyl esters (where R₇ & R₈ are as defined earlier) using suitable base such as K₂CO₃, Cs₂CO₃, KOH, NaH afforded ester compound 6. Compound of formula 6 was reacted with suitable aromatic acids or suitable aromatic acid chlorides and appropriate acylating agents to obtain compound of Formula 7. Deprotection and hydrolysis of compound of formula 7, using suitable reagents & techniques as is known in the art, gives compound of formula (I)

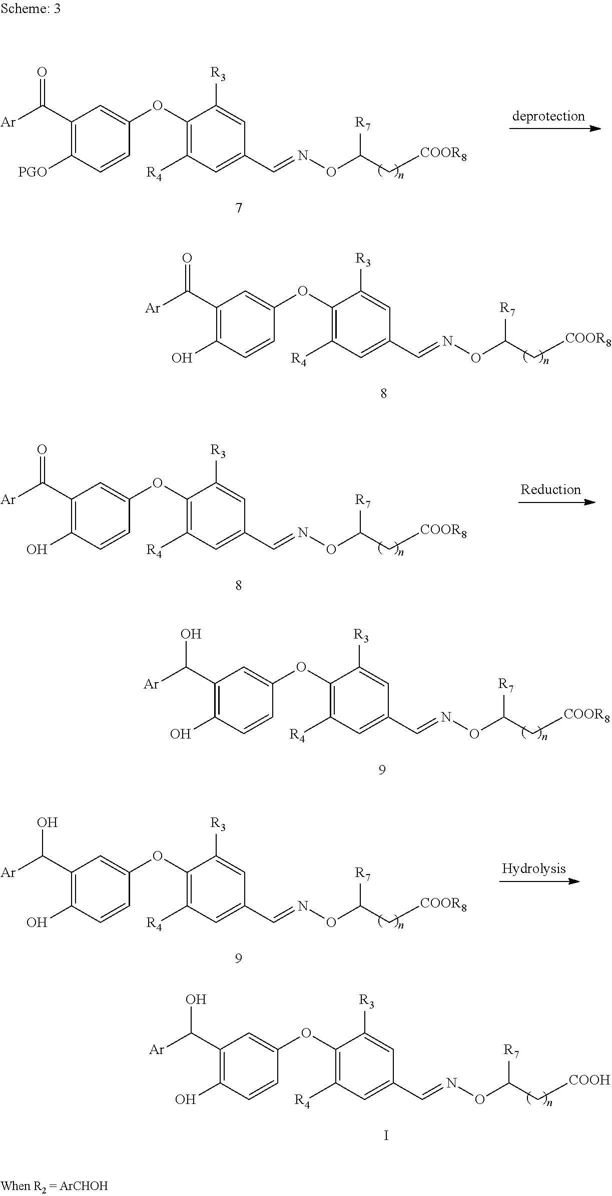

Starting from compound of formula 7 (Scheme 2) deprotection and then reduction of carbonyl group using suitable reducing agents like NaBH₄, LAH and the like in suitable solvents like THF, Diethyl ether etc. to afford compound of formula 9. Finally hydrolysis of compound of formula 9, using suitable reagents & techniques as is known in the art, gives compound of formula (I)

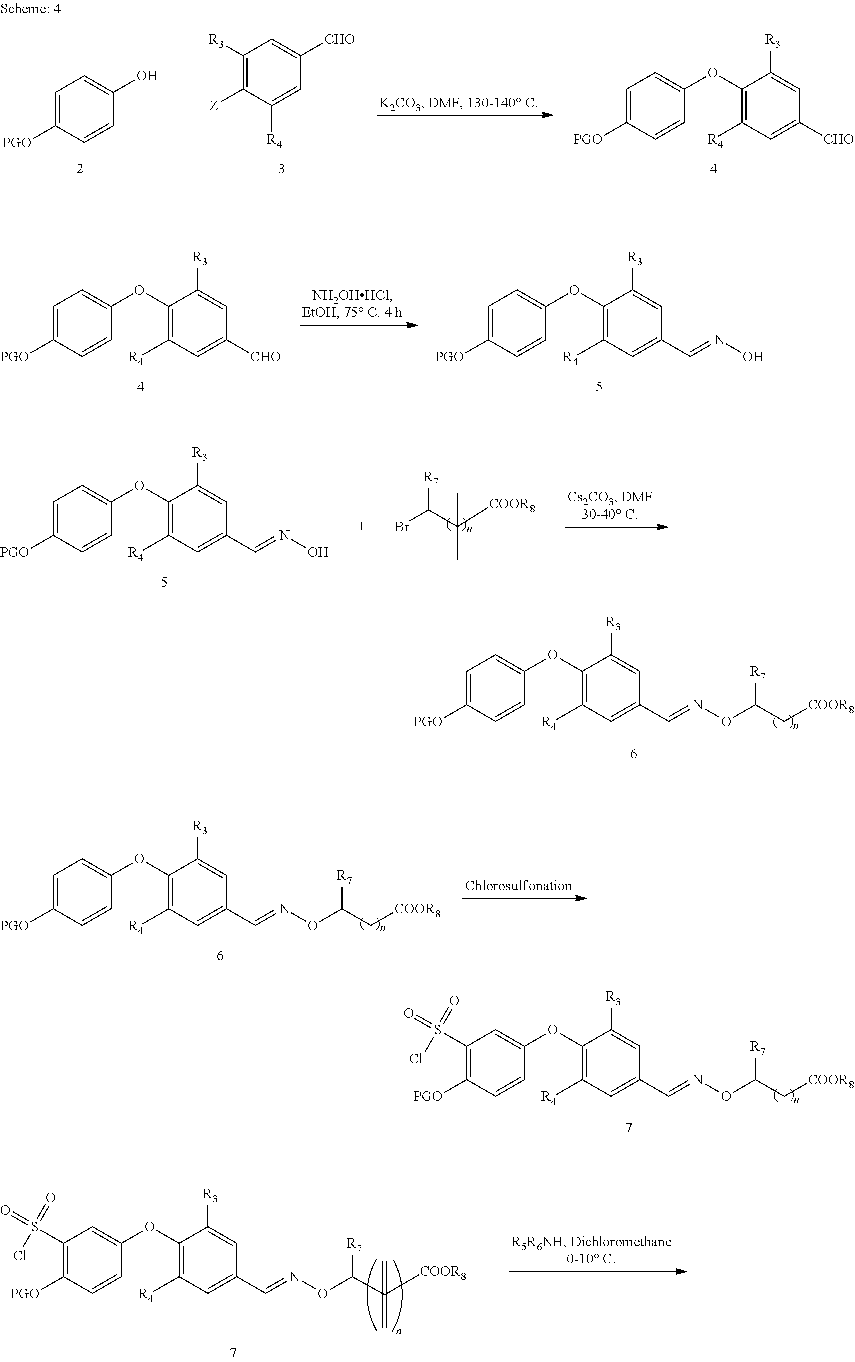

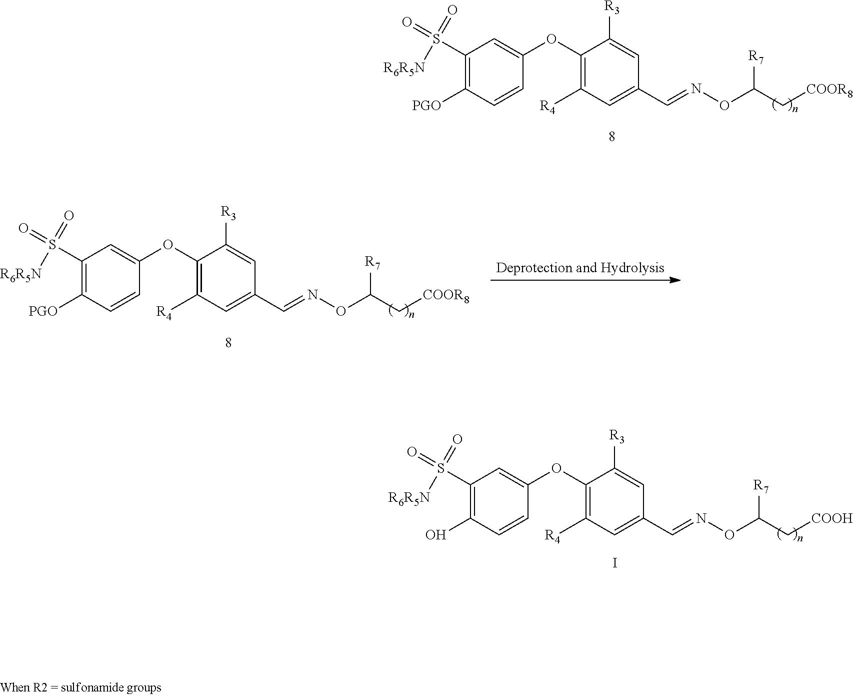

Reacting protected phenol of formula 2, wherein PG represents suitable protecting groups known to persons skilled in the art (for e.g. those described in T. W. Greene and P. G. M. Wuts “Protective groups in Organic Synthesis”, John Wiley & Sons, Inc, 1999, 3^{rd} Ed., 201-245 along with references therein), with aldehyde compound of formula 3 wherein R₃ and R₄ are as defined earlier and Z is halogen to give coupled product of formula 4. The reaction may be carried out in solvents such as DMF, DMSO, THF, toluene and the like or their suitable mixtures, in the presence of a base such as K₂CO₃, NaH, KOH and the like or their suitable mixtures. Reaction of coupled product 4 with hydroxyl amine hydrochloride afforded oxime compound of formula 5. Alkylation of the oxime compound of formula 5 with bromo alkyl esters (where R₇ & R₈ are as defined earlier) using suitable base such as K₂CO₃, Cs₂CO₃, KOH, NaH and the like afforded ester compound 6. Compound of formula 6 was reacted with chlorosulfonic acid at suitable temp. to give chlorosulfonated product of formula 7 which was then reacted with suitable aliphatic or aromatic amine R₅R₆NH, (where R₅ & R₆ are as defined earlier) to afford compound of formula 8, on deprotection and hydrolysis of compound 8 gives compound of formula (I).

The invention is explained in greater detail by the examples given below, which are provided by way of illustration only and therefore should not be construed to limit the scope of the invention.

1H NMR spectral data given in the examples (vide infra) are recorded using either a 300 MHz spectrometer (Bruker AVANCE-300) or a 400 MHz spectrometer (Bruker Avance2) and reported in δ scale. Until and otherwise mentioned the solvent used for NMR is CDCl₃ using tetramethyl silane as the internal standard.

### EXAMPLE 1

### Preparation of 2-(((3,5-dichloro-4-(4-hydroxy-3-isopropyl phenoxy)benzylidene)amino)oxy)acetic acid

### Step 1: 4-(3-isopropyl-4-methoxyphenoxy)-3,5-dichlorobenzaldehyde

To a solution 3-isopropyl-4-methoxyphenol (0.37 g, 2.22 mmol) in DMF (3.7 mL) was added K₂CO₃ (0.50 g, 3.64 mmol) and 3,5-dichloro-4-iodobenzaldehyde (0.61 g, 2.02 mmol). The reaction was stirred at 130-135° C. for 5 hrs. The reaction mixture was poured over ice. The product was taken up in ethyl acetate, washed with water, brine, dried over sodium sulphate, filtered and concentrated to give the crude product, which was purified by column chromatography over flash silica gel (hexane:ethylacetate 90:10) to afford pure 4-(3-isopropyl-4-methoxyphenoxy)-3,5-dichlorobenzaldehyde. (0.2g, 30% yield)

¹H NMR:(CDCl₃, 400 MHz): 1.18(6H, d, J=6.8 Hz), 3.27-3.30(1H, m), 3.79(3H, s), 6.44-6.47 (1H, dd, J=3.2&9.2 Hz), 6.70(1H, d, J=8.8 Hz), 6.85(1H, d, J=3.2 Hz), 7.91(2H, s).

### Step 2: 4-(3-isopropyl-4-methoxyphenoxy)-3,5-dichlorobenzaldehyde oxime

A mixture of 4-(3-isopropyl-4-methoxyphenoxy)-3,5-dichlorobenzaldehyde (0.2 g, 0.589 mmol) in EtOH (0.4 mL) and H₂O (0.6 mL) and Hydroxyl amine hydrochloride (0.04 g, 0.589 mmol) was heated at 75° C. for 3 hrs. The reaction mixture was poured over ice. The product was taken up in, ethyl acetate, washed with water, brine, dried over sodium sulphate, filtered and concentrated to give the crude product, which was purified by column chromatography over flash silica gel (hexane:ethyl acetate 90:10) to afford pure 4-(3-isopropyl-4-methoxyphenoxy)-3,5-dichlorobenzaldehyde oxime (0.19 g, 91%).

¹H NMR: (CDCl₃, 400 MHz): 1.18(6H, d, J=7.2 Hz), 3.24-3.31(1H, m), 3.78(3H, s), 6.44-6.47 (1H, dd, J=2.8&8.8 Hz), 6.70(1H, d, J=8.8 Hz), 6.84(1H, d, J=2.8 Hz), 7.61(214, s), 8.05(1H, s).

### Step 3: Ethyl 2-(((3,5-dichloro-4-(3-isopropyl-4-methoxy phenoxy)benzylidene)amino)oxy)acetate

To a solution of 4-(3-isopropyl-4-methoxyphenoxy)-3,5-dichlorobenzaldehyde oxime (0.19 g, 0.536 mmol) in DMF (1.5 mL) was added Cs₂CO₃ (0.26 g, 0.805 mmol). To that added Ethyl bromo acetate (0.1 g, 0.59 mmol) and the reaction mixture was stirred at 20-25° C. for 3 hrs. The reaction mixture was poured over ice. The product was taken up in ethyl acetate, washed with water, brine, dried over sodium sulphate, filtered and concentrated to give the crude product, which was purified by column chromatography over flash silica gel (hexane:ethylacetate 90:10) to afford pure Ethyl 2-(((3,5-dichloro-4-(3-isopropyl-4-methoxy phenoxy)benzylidene)amino)oxy)acetate as an oil (0.19 g, 80%).

¹H NMR:(CDCl₃, 400 MHz): 1.18(6H, d, J=6.8 Hz), 1.27-1.29(3H, m),3.24-3.31(1H, m), 3.78 (3H, s), 4.24-4.29(2H, m), 4.72(2H, s), 6.3-6.46(1H, dd, J=3.2&8.8 Hz), 6.70(1H, d, J=9.2 Hz), 6.83 (1H, d, J=3.2 Hz), 7.61(2H, s), 8.12 (1H, s).

### Step 4: 2-(((3,5-dichloro-4-(4-hydroxy-3-isopropyl phenoxy)benzylidene)amino)oxy)acetic acid

To a solution of Ethyl 2-(4-(3-isopropyl-4-methoxyphenoxy)-3,5-dichloro benzylidene aminooxy)acetate (0.19 g, 0.431 mmol) in dichloromethane (1.9 mL) was cooled to −60 to −70° C. under N₂ atomsphere. To that 1M BBr₃ solution in dichloromethane (1.72 mL) was added dropwise. The reaction mixture was allowed to warm up to 20-25° C. over 5 h. then diluted with more CH₂Cl₂ (25 mL) and quenched with H₂O. After stirring at 20-25° C. for 30 min, organic phase was separated, washed with water, brine, dried over sodium sulphate, filtered and concentrated to give crude product. The crude product was purified by column chromatography over flash silica gel (chloroform:methanol) gradient elution from 95:5 to 90:10 to give pure 2-(((3,5-dichloro-4-(4-hydroxy-3-isopropyl phenoxy)benzylidene)amino)oxy)acetic acid (0.065 g, 38%).

¹H NMR:(DMSO-D6, 400 MHz): 1.10(6H, d, J=6.8 Hz), 3.16-3.17(1H, m), 4.65(2H, s), 6.30-6.33(1H, dd, J=3.2&8.8 Hz), 6.64-6.69(2H, m), 7.80(2H, s), 8.34(1H, s).

Using appropriate starting materials and suitable modifications of one or more of the process steps described above, either alone or in suitable combination, including suitable addition and/or deletion of steps as may be necessary, & which are well within the scope of a person skilled in the art, the following compounds were prepared in an analogous manner

### EXAMPLE 2

### 2-(((4-(3-(sec-butyl)-4-hydroxyphenoxy)-3,5-dichlorobenzylidene)amino)oxy)acetic acid

¹H NMR:(CDCl₃, 400 MHz): 0.85(3H, t, J=7.4 Hz), 1.19(3H, d, J=6.8 Hz), 1.51-1.63(2H, m), 2.89-2.95(1H, m), 4.79(2H, s), 6.40-6.43(1H, dd, J=2.8&8.8 Hz), 6.64(1H, d, J=8.8 Hz), 6.72(1H, d, J=2.8 Hz), 7.60(2H, s), 8.11(1H, s). % Yield: 61%

### EXAMPLE 3

### 2-(((3,5-dibromo-4-(4-hydroxy-3-isopropylphenoxy)benzylidene)amino)oxy)acetic acid

¹H NMR:(CDCl₃, 400 MHz): 1.22(6H, d, J=6.8 Hz), 3.13-3.20(1H, m), 4.79(2H, s), 6.36-6.39 (1H, dd, J=3.2&8.8 Hz), 6.63(1H, d, J=8.8 Hz), 6.79(1H, d, J=2.8 Hz), 7.82(2H, s), 8.12(1H, s). % Yield: 66%

### EXAMPLE 4

### 2-(((3,5 -dichloro-4-((6-hydroxy-[1,1′-biphenyl]-3-yl)oxy)benzylidene)amino)oxy)acetic acid

¹H NMR:(CD₃OD, 400 MHz): 4.57(2H, s), 6.58-6.61(1H, dd, J=3.2&8.8 Hz), 6.70(1H, d, J=3.2 Hz), 6.81(1H, d, J=8.8 Hz), 7.28(1H, d, J=7.6 Hz), 7.36(2H, t, J=7.6 Hz), 7.49(2H, d, J=7.2 Hz), 7.73(2H, s), 8.16(11-1, s). % Yield: 13%

### EXAMPLE 5

2-(((4-(3-benzyl-4-hydroxyphenoxy)-3,5-dichlorobenzylidene)amino)oxy)acetic acid

¹H NMR:(CD₃OD, 400 MHz): 3.87(2H, s), 4.71(2H, s), 6.42-6.43(1H, m), 6.46(1H, d, J=2.8 Hz), 6.69(1H, d, J=8.4 Hz), 7.11-7.16(3H, m), 7.19-7.23(2H, m), 7.68(2H, s), 8.18(1H, s). % Yield: 55%

### EXAMPLE 6

### 2-(((3,5-dibromo-4-(3 -(sec-butyl)-4-hydroxyphenoxy)benzylidene)amino)oxy)acetic acid

¹H NMR:(CDCl₃, 400 MHz): 0.86(3H, t, J=7.2 Hz), 1.17(3H, d, J=6.8 Hz), 1.48-1.62(2H, m), 2. 97-3.02(1H, m), 4.68(2H, s), 6.36-6.39(1H, dd, J=2.4&8.4 Hz), 6.64(1H, d, J=8.4 Hz), 6.68(1H, d, J=2.8 Hz), 7.80(214, s), 8.10(1H, s). % Yield: 29%

### EXAMPLE 7

### 2-(((3,5-dibromo-4-((6-hydroxy-[1,1′-biphenyl]-3-yl)oxy)benzylidene)amino)oxy)acetic acid

¹H NMR: (CDCl₃, 400 MHz): 4.79(2H, s), 6.69-6.75(2H, m), 6.91(1H, d; J=8.8 Hz), 7.45-7.48 (5H, m), 7.82(2H, s), 8.11(1H, s). % Yield: 23%

### EXAMPLE 8

### 2-(((3,5-dichloro-4-(4-hydroxy-3-isopropylphenoxy)benzylidene)amino)oxy)propanoic acid

¹H NMR:(CDCl₃, 400 MHz): 1.22(6H, d, J=7.2 Hz), 1.60(3H, d, J=6.8 Hz), 3.15-3.18(114, m), 4.88-4.90(1H, m), 6.37-6.40(1H, dd, J=2.8 &8.8 Hz), 6.62(1H, d, J=8.8 Hz), 6.81(1H, d, J=3.2 Hz), 7.60(2H, s), 8.09(1H, s). % Yield: 59%

### EXAMPLE 9

### 2-(((4-(3-benzyl-4-hydroxyphenoxy)-3,5-dibromobenzylidene)amino)oxy)acetic acid

¹H NMR:(CDCl₃, 400 MHz): 3.93(2H, s), 4.77(2H, s), 6.66(1H, d, J=3.2 Hz), 6.69-6.71(2H, m), 7.18-7.30(5H, m), 7.79(2H, s), 8.10 (1H, s). % Yield: 58%

### EXAMPLE 10

### 2-(((3,5-dibromo-4-(4-hydroxy-3-isopropylphenoxy)benzylidene)amino)oxy)propanoic acid

¹H NMR:(CDCl₃, 400 MHz): 1.22(6H, d, J=7.2 Hz), 1.60(3H, d, J=6.8 Hz), 3.08-3.20(1H, m), 4.86-4.91(1H, q, J=7.2 Hz), 6.34-6.37(1H, dd, J=3.2&8.8 Hz), 6.62(1H, d, J=8.8 Hz), 6.80(1H, d, J=3.2 Hz), 7.80(2H, s), 8.07(111, s). % Yield: 43%

### EXAMPLE 11

### 2-(((3,5-dibromo-4-((6-hydroxy-[1,1′-biphenyl]-3-yl)oxy)benzylidene)amino)oxy)propanoic acid

¹H NMR:(CDCl₃, 400 MHz): 1.59(3H, d, J=7.2 Hz), 4.85-4.91(1H, q, J=6.8&7.2 Hz), 6.69(1H, d, J=2.8 Hz), 6.72-6.75(1H, d, J=3.2&8.8 Hz), 6.91(1H, d, J=8.4 Hz), 7.37-7.52(5H, m), 7.80(2H, s), 8.01(1H, s). % Yield: 90%

### EXAMPLE 12

### 2-(((3,5-dibromo-4-(4-hydroxy-3-isopropylphenoxy)benzylidene)amino)oxy)-2-methylpropanoic acid

¹H NMR:(CDCl₃, 400 MHz): 1.22(6H, d, J=7.2 Hz), 1.63(6H, s), 3.13-3.20(1H, m), 6.36-6.38 (1H, dd, J=3.2&8.8 Hz), 6.62(1H, d, J=8.4 Hz), 6.80(1H, d, J=3.2 Hz), 7.80(2H, s), 8.05(1H, s). % Yield: 96%

### EXAMPLE 13

### 2-(((3,5-dibromo-4-(4-hydroxy-3-isopropylphenoxy)benzylidene)amino)oxy)butanoic acid

¹H NMR:(DMSO-D6, 400 MHz): 0.95(3H, t, J=6.4 Hz), 1.10(6H, d, J=7.2 Hz), 1.77-1.86(2H, m), 3.10-3.17(1H, m), 4.56-4.60(1H, m), 6.26-6.29(1H, dd, J=3.2 &8.8 Hz), 6.64-6.66(2H, m), 7.95 (2H, s), 8.33(1H, s). % Yield: 84%

### EXAMPLE 14

### 2-(((3,5-dibromo-4-(3-(sec-butyl)-4-hydroxyphenoxy)benzylidene)amino)oxy)propanoic acid

¹H NMR:(CDCl₃, 400 MHz): 0.85(3H, t, J=7.2 Hz), 1.26(314, d, J=7.2 Hz), 1.41-1.66(5H, m), 2.88-2.95(1H, m), 4.85-4.91(1H, q, J=7.2 Hz), 6.38-6.41(1H, dd, J=2.8 Hz&8.8 Hz), 6.64(1H, d, J=8.8 Hz), 6.71(1H, d, J=2.8 Hz), 7.80(2H, s), 8.08(1H, s). % Yield: 10%

### EXAMPLE 15

### 2-(((3,5-dibromo-4-(3-(sec-butyl)-4-hydroxyphenoxy)benzylidene)amino)oxy)-2-methyl propanoic acid

¹H NMR:(DMSO-D₆, 400 MHz): 0.76(3H, t, J=7.2 Hz), 1.06(3H, d, J=6.8 Hz), 1.41-1.54(8H, m), 2.90-2.96(1H, m), 6.31-6.33(1H, dd, J=2.8&8.8 Hz), 6.58(1H, d, J=2.4 Hz), 6.66(1H, d, J=8.8 Hz), 7.93(2H, s), 8.25(1H, s). % Yield: 87%

### EXAMPLE 16

### 2-(((4-(3-benzyl-4-hydroxyphenoxy)-3,5-dibromobenzylidene)amino)oxy)propanoic acid

¹H NMR:(DMSO-D₆, 400 MHz): 1.41(3H, d, J=6.8 Hz), 3.80(2H, s), 4.72(1H, q, J=6.8 Hz), 6.37-6.40(1H, dd, J=2.8&5.6 Hz), 6.57(1H, d, J=3.2 Hz), 6.69(1H, d, J=8.8 Hz), 7.11-7.25(5H, m), 7.93(2H, s), 8.30(1H, s). % Yield: 54%

### EXAMPLE 17

### 2-(((4-(3-benzyl-4-hydroxyphenoxy)-3,5-dibromobenzylidene)amino)oxy)butanoic acid

¹H NMR:(CDCl₃, 400 MHz): 0.95(3H, m), 1.05-1.20(2H, m), 3.93(2H, s), 4.73(1H, m), 6.49-6.51(1H, m), 6.69(2H, m), 7.14-7.34(5H, m), 7.78(2H, s), 8.10(1H, s) % Yield: 58%

### EXAMPLE 18

### 2-(((3,5-dibromo-4-(3-(sec-butyl)-4-hydroxyphenoxy)benzylidene)amino)oxy)-2-phenylacetic acid

¹H NMR:(DMSO-D₆, 400 MHz): 0.76(3H, t, J=7.2 Hz), 1.06(3H, d, J=6.8 Hz), 1.41-1.52(2H, m), 2.90-2.96(1H, m), 5.69(1H, s), 6.31-6.34(1H, dd, J=2.8&8.8 Hz), 6.57(1H, d, J=2.8 Hz), 6.66 (1H, d, J=8.8 Hz), 7.40-7.45(3H, m), 7.47-7.5(2H, m), 7.96(2H, s), 8.40(1H, s). % Yield: 53%

### EXAMPLE 19

### 2-(((3,5-dibromo-4-(3-(sec-butyl)-4-hydroxyphenoxy)benzylidene)amino)oxy)butanoic acid

¹H NMR:(CD₃OD, 400 MHz): 0.81(3H, t, J=7.4 Hz), 1.06(3H, t, J=7.6 Hz), 1.12(3H, d, J=7.2 Hz), 1.50-1.56(2H, m), 1.89-1.95(2H, m), 2.99-3.03(1H, m), 4.67(1H, t, J=6.2 Hz), 6.35-6.38(1H, dd, J=2.8&8.4 Hz), 6.56(1H, d, J=2.8 Hz), 6.65(1H, d, J=8.4 Hz), 7.91(2H, s), 8.18(1H, s). % Yield: 69%

### EXAMPLE 20

### 2-(((3,5-dibromo-4-((6-hydroxy-[1,1′-biphenyl]-3-yl)oxy)benzylidene)amino)oxy)-2-methyl propanoic acid

¹H NMR:(CD₃OD, 400 MHz): 1.56(6H, s), 6.57-6.60(1H, dd, J=3.2 &8.8 Hz), 6.67(1H, d, J=3.2 Hz), 6.81(1H, d, J=9.2 Hz), 7.27(1H, t, J=7.2 Hz), 7.36(2H, t, J=7.6 Hz), 7.49(2H, d, J=7.2 Hz), 7.91(2H, s), 8.11(1H, s). % Yield: 49%

### EXAMPLE 21

### 2-(((3,5-dibromo-4-((6-hydroxy-[1,1′-biphenyl]-3-yl)oxy)benzylidene)amino)oxy)butanoic acid

¹H NMR:(DMSO-D₆, 400 MHz): 0.95(3H, t, J=7.6 Hz), 1.77-1.86(2H, m), 4.57(1H, t, J=6 Hz), 6.59-6.62(1H, dd, J=3.2&8.8 Hz), 6.68(1H, d, J=3.2 Hz), 6.87(1H, d, J=8.8 Hz), 7.27(1H, t, J=7.2 Hz), 7.37(2H, t, J=7.2 Hz), 7.47(2H, d, J=7.6 Hz), 7.96(2H, s), 8.33(1H, s). % Yield: 58%

### EXAMPLE 22

### 2-(((4-(3-benzyl-4-hydroxyphenoxy)-3,5-dibromobenzylidene)amino)oxy)-2-methylpropanoic acid

¹H NMR:(DMSO-D6, 400 MHz): 1.46(6H, s), 3.80(2H, s), 6.38-6.41(1H, dd, J=3.2&8.8 Hz), 6.56(1H, d, J=3.2 Hz),6.70(1H, d, J=8.8 Hz), 7.11-7.25(5H, m), 7.91(2H, s), 8.23(1H, s). % Yield: 50%

### EXAMPLE 23

### 2-(((4-(3-(sec-butyl)-4-hydroxyphenoxy)-3,5-dichlorobenzylidene)amino)oxy)propanoic acid

¹H NMR:(CDCl₃, 400 MHz): 0.85(3H, t, J=7.6 Hz), 1.19(3H, d, J=6.8 Hz), 1.52-1.60(2H, m), 1.64(3H, d, J=7.2 Hz), 2.90-2.97(1H, m), 4.87-4.91(1H, m), 6.42(1H, dd, J=2.8 Hz&8.4 Hz), 6.63 (1 H, d, J=8.4 Hz), 6.74(111, s), 7.59(2H, s), 8.08(1H, s). % Yield: 45%

### EXAMPLE 24

### 2-(((3,5-dibromo-4-(3-(tert-butyl)-4-hydroxyphenoxy)benzylidene)amino)oxy)propanoic acid

¹H NMR:(CDCl₃, 400 MHz): 1.38(9H, s), 1.66(3H, d, J=7.2 Hz), 4.88(1H, q, J=7.2 Hz), 6.32-6.34(1H, dd, J=3.2 Hz & 8.8 Hz), 6.53(1H, d, J=8.8 Hz), 6.92(1H, d, J=3.2 Hz), 7.80(2H, s), 8.08 (1H, s). % Yield: 30%

### EXAMPLE 25

### 2-(((3,5-dibromo-4-(3-ethyl-4-hydroxyphenoxy)benzylidene)amino)oxy)propanoic acid

¹H NMR:(CDCl₃, 400 MHz): 1.21(3H, t, J=7.2 Hz), 1.60(3H, d, J=6.8 Hz), 2.56-2.62(2H, q, J=7.2&7.6 Hz), 4.86-4.91(1H, q, J=6.8&7.2 Hz), 6.42-6.45(1H, dd, J=2.8&8.8 Hz), 6.65(1H, d, J=8.4 Hz), 6.68(1H, d, J=2.8 Hz), 7.81(2H, s), 8.18(1H, s). % Yield: 50%

### EXAMPLE 26

### Preparation of 2-4(3,5-dibromo-4-(3-(4-chlorobenzoyl)-4-hydroxy phenoxy)benzylidene)amino)oxy)propanoic acid

### Step 1: 3,5-dibromo-4-(4-methoxyphenoxy)benzaldehyde

To a solution of 4-methoxyphenol (1.6 g, 12.9 mmol) in DMF (16 mL) was added K₂CO₃ (3.25 g, 25.8 mmol) and 3,5-bromo-4-iodobenzaldehyde (4.6 g, 12.9 mmol). The reaction was stirred at 130-135° C. for 2 hrs. The reaction mixture was poured over ice. The product was taken up in ethyl acetate, washed with water, brine, dried over sodium sulphate, filtered and concentrated to give the crude product, which was purified by column chromatography over flash silica gel (hexane:ethyl acetate 90:10) to afford pure 3,5-dibromo-4-(4-methoxyphenoxy)benzaldehyde (2.0 g, 44% yield)

### Step 2: 3,5-dibromo-4-(4-methoxyphenoxy)benzaldehyde oxime

A mixture of 3,5-dibromo-4-(4-methoxyphenoxy)benzaldehyde (2.0 g, 5.18 mmol) in EtOH (14 mL) and H₂O (14 mL) and hydroxyl amine hydrochloride (1.44 g, 20.74 mmol) was heated at 90° C. for 2 hrs. The reaction mixture was poured over ice. The product was taken up in ethyl acetate, washed with water, brine, dried over sodium sulphate, filtered and concentrated to give the crude product, which was purified by column chromatography over flash silica gel (hexane:ethyl acetate 90:10) to afford pure 3,5-dibromo-4-(4-methoxyphenoxy)benzaldehyde oxime (1.92 g, 96%).

¹H NMR:(CDCl₃, 400 MHz): 3.77(3H, s), 6.74-6.76 (2H, m), 6.82-6.89 (2H, m), 7.82 (2H, s), 8.03 (1H, s).

### Step 3: Ethyl 2-(((3,5-dibromo-4-(4-methoxyphenoxy)benzylidene)amino)oxy)propanoate

To a solution of 3,5-dibromo-4-(4-methoxyphenoxy)benzaldehyde oxime (0.6 g, 1.496 mmol) in DMF (3.75 mL) was added Cs₂CO₃ (0.73 g, 2.24 mmol). To that was added Ethyl-2-bromo propanoate (0.29 g, 1.64 mmol) and then reaction mixture was stirred at 20-25° C. for 2 hrs. The reaction mixture was poured over ice. The product was taken up in ethyl acetate, washed with water, brine, dried over sodium sulphate, filtered and concentrated to give the crude product, which was purified by column chromatography over flash silica gel (hexane:ethyl acetate 95:05) to afford pure ethyl 2-(((3,5-dibromo-4-(4-methoxyphenoxy)benzylidene)amino)oxy)propanoate as an oil (0.68 g, 90%).

¹H NMR: (CDCl₃, 400 MHz): 1.30-1.35(3H, m), 1.51-1.53(3H, m), 3.77(3H, s), 4.22-4.28(2H, q, J=7.2 Hz), 4.79-4.85(1H, q, J=7.2 Hz), 6.74(2H, d, J=9.2 Hz), 6.82(2H, d, J=9.2 Hz), 7.79(2H, s), 8.07(1H, s).

### Step 4: Ethyl 2-(((3,5-dibromo-4-(3-(4-chlorobenzoyl)-4-methoxy phenoxy)benzylidene)amino)oxy)propanoate

A mixture of ethyl 2-(((3,5-dibromo-4-(4-methoxyphenoxy)benzylidene)amino)oxy)propanoate (0.5 g, 0.998 mol) and 4-chlorobenzoic acid (0.31 g, 1.99 mmol) in Eaton's reagent (6.33 mL) was heated at 95° C. for 16 h. The reaction mixture was poured over ice. The product was taken up in ethyl acetate, washed with water, brine, dried over sodium sulphate, filtered and concentrated to give the crude product, which was purified by column chromatography over flash silica gel (hexane:ethyl acetate 95:05) to afford pure ethyl 2-(((3,5-dibromo-4-(3-(4-chlorobenzoyl)-4-methoxy phenoxy)benzylidene)amino)oxy)propanoate (0.143 g, 28%).

¹H NMR: (CDCl₃, 400 MHz): 1.22-1.27(3H, m), 1.45-1.58(3H, m), 3.69(3H, s), 4.23-4.28(2H, m), 4.81-4.87(1H, m), 6.82(1H, d, J=2.8 Hz), 6.91-6.98(2H, m), 7.24(1H, m), 7.39-7.42(2H, d, J=8.4 Hz), 7.73-7.77(3H, m), 8.19(1H, s)

### Step 5: Ethyl 2-(((3,5-dibromo-4-(3-(4-chlorobenzoyl)-4-hydroxy phenoxy)benzylidene)amino)oxy)propanoate

A solution of ethyl 2-(((3,5-dibromo-4-(3-(4-chlorobenzoyl)-4-hydroxy phenoxy)benzylidene)amino)oxy)propanoate (0.143 g, 0.223 mmol) in dichloromethane (1.4 mL) was cooled to −60 to −70° C. under N₂ atomsphere. To that 1M BBr₃ solution in dicloromethane (0.89 mL) was added dropwise. The reaction mixture was allowed to warm up to −20° C. over 2 h. Then diluted with more CH₂Cl₂ (25 mL) and quenched with H₂O. After stirring at 20-25° C. for 10 min, organic phase was separated, washed with water, brine, dried over sodium sulphate, filtered and concentrated to give crude product. The crude product was purified by column chromatography over flash silica gel (hexane:ethylacetate 95:05) to give pure ethyl 2-(((3,5-dibromo-4-(3-(4-chlorobenzoyl)-4-hydroxy phenoxy)benzylidene)amino)oxy)propanoate. (0.139 g, 100%).

¹H NMR: (CDCl₃, 400 MHz): 1.22-1.27 (3H, m), 1.45-1.58(314, m), 4.23-4.28(2H, m), 4.81-4.87 (1H, m), 6.97-7.06(3H, m), 7.44(2H, d, J=8.8 Hz), 7.63(2H, d, J=8.4 Hz), 7.77(2H, s), 8.19(1H, s).

### Step 6: 2-(((3,5-dibromo-4-(3-(4-chlorobenzoyl)-4-hydroxyphenoxy)benzylidene)amino)oxy)propanoic acid

The ester obtained from step 5 above (0.139 g, 0.22 mmol) was dissolved in EtOH (0.84 mL) and to that solution of NaOH (0.011 g, 0.29 mmol) in H₂O (0.42 mL) was added and it was stirred at 50° C. for 2 hr. Ethanol was evaporated from the reaction mixture and H₂O was added & washed with diethyl ether. The aqueous layer was acidified to pH 4 using 10% HCl solution and extracted with ethyl acetate. The organic layer was washed with water, brine, dried over sodium sulphate, filtered and concentrated to give pure product 2-(((3,5-dibromo-4-(3-(4-chlorobenzoyl)-4-hydroxyphenoxy)benzylidene)amino)oxy)propanoic acid (0.04 g, 30%)

¹H NMR: (CD₃OD, 400 MHz): 1.52 (3H, d, J=6.8 Hz), 4.79-4.81(1H, m), 6.76(1H, d, J=2.8 Hz), 7.02 (1H, d, J=8.8 Hz), 7.12-7.15(1H, dd, J=3.2&9.2 Hz), 7.49(2H, d, J=8.4 Hz), 7.66(2H, d, J=8.8 Hz), 7.92(2H, s), 8.14(1H, s).

### EXAMPLE 27

### 2-(((3,5-dibromo-4-(3-((4-chlorophenyl)(hydroxy)methyl)-4-hydroxy phenoxy)benzylidene)amino)oxy)propanoic acid

To a solution of ethyl 2-(((3,5-dibromo-4-(3-(4-chlorobenzoyl)-4-hydroxy phenoxy)benzylidene)amino)oxy)propanoate (0.25 g, 0.39 mmol Example 26, step 5 above) in MeOH (2.5 mL) was added NaBH₄ (13mg, 0.35 mmol) at 0-10° C. The reaction was stirred at same temperature for 2-3 hrs. The product was taken up in ethyl acetate, washed with water, brine, dried over sodium sulphate, filtered and concentrated to give the crude product, which was purified by column chromatography over flash silica gel (hexane:ethyl acetate 90:10) to afford pure ethyl 2-(((3,5-dibromo-4-(3-((4-chlorophenyl)(hydroxy)methyl)-4-hydroxy phenoxy)benzylidene)amino)oxy)propanoate (0.25 g, 100%) which was hydrolyzed similar to the procedure given in (Example 26, step 6) to afford 2-(((3,5-dibromo-4-(3-((4-chlorophenyl)(hydroxy)methyl)-4-hydroxy phenoxy)benzylidene)amino)oxy)propanoic acid (0.113 g, 62%)

¹H NMR: (CD₃OD, 400 MHz): 1.50(3H, d, J=7.2 Hz), 4.77-4.79(1H, m), 6.00(1H, s), 6.53-6.56 (1H, dd, J=3.2&8.8 Hz), 6.68(1H, d, J=8.8 Hz), 6.79(1H, d, J=2.8 Hz), 7.25(2H, d, J=8.8 Hz), 7.31 (2H, d, J=8.4 Hz), 7.90(2H, s), 8.15(1H, s).

Using appropriate starting materials and suitable modifications of one or more of the processes described above, either alone or in suitable combination of the steps disclosed therein, including suitable addition and/or deletion of steps as may be necessary, well within the scope of a person skilled in the art, the following compounds (Examples 28-35) were prepared in an analogous manner to that of Examples 26 & 27

### EXAMPLE 28

### 2-(((3,5-dibromo-4-(3-(4-chlorobenzoyl)-4-hydroxyphenoxy)benzylidene)amino)oxy)acetic acid

¹H NMR:(DMSO-D₆, 400 MHz): 4.63(2H, s), 6.74(1H, m), 6.92(2H, m), 7.57(2H, d, J=8.4 Hz), 7.68(2H, d, J=8.8 Hz), 7.97(2H, s), 8.32(1H, s). % Yield: 9%

### EXAMPLE 29

### 2-(((3,5-dibromo-4-(3-((4-chlorophenyl)(hydroxy)methyl)-4-hydroxyphenoxy)benzylidene)amino)oxy)acetic acid

¹H NMR:(DMSO-D₆, 400 MHz): 4.57(2H, s), 5.82-5.85(1H, m), 6.46-6.49(1H, dd, J=3.2&8.8 Hz), 6.67(1H, d, J=8.8 Hz), 6.88(1H, d, J=3.2 Hz), 7.30(4H, m), 7.96(2H, s), 8.31(1H, s). % Yield: 58%

### EXAMPLE 30

### 2-(((3,5-dibromo-4-(3-(3-chlorobenzoyl)-4-hydroxyphenoxy)benzylidene)amino)oxy)propanoic acid

¹H NMR: (DMSO-D₆, 400 MHz): 1.41(3H, d, J=6.8 Hz), 4.71-4.72(1H, m), 6.74(1H, d, J=3.2 Hz), 6.93-6.99(2H, m), 7.54(1H, d, J=8.0 Hz), 7.61-7.62(2H, m), 7.69(1H, d, J=8.8 Hz), 7.96(2H, s), 8.30(1H, s). % Yield: 24%

### EXAMPLE 31

### 2-(((3,5-dibromo-4-(3-(4-bromobenzoyl)-4-hydroxyphenoxy)benzylidene)amino)oxy)acetic acid

¹H NMR: (DMSO-D₆, 400 MHz): 4.63(2H, s), 6.74(1H, m), 6.92(2H, m), 7.60(2H, d, J=8.8 Hz), 7.72(2H, d, J=8.4 Hz), 7.97(2H, s), 8.32 (1H, s). % Yield: 8%

### EXAMPLE 32

### 2-(((3,5-dibromo-4-(3-((4-bromophenyl)(hydroxy)methyl)-4-hydroxyphenoxy)benzylidene)amino)oxy)acetic acid

¹H NMR: (DMSO-D₆, 400 MHz): 4.61(2H, s), 5.82(1H, s), 6.46-6.49(1H, dd, J=3.2&8.8 Hz), 6.67(1H, d, J=8.8 Hz), 6.88(1H, d, J=3.2 Hz), 7.24(2H,d, J=8.4 Hz), 7.44(2H, d, J=8.4 Hz), 7.97 (2H, s), 8.32(1H, s). % Yield: 60%

### EXAMPLE 33

### 2-(((3,5-dibromo-4-(3-(3-chlorobenzoyl)-4-hydroxyphenoxy)benzylidene)amino)oxy)acetic acid

¹H NMR: (DMSO-D₆, 400 MHz): 4.58(2H, s), 6.72(1H, d, J=2.8 Hz), 6.94-6.98(2H, m), 7.53(1H, m), 7.61-7.62(2H, m), 7.69(1H, d, J=8.4 Hz), 7.97(2H, s), 8.34(1H, s). % Yield: 16%

### EXAMPLE 34

### 2-(((3,5-dibromo-4-(3-((3-chlorophenyl)(hydroxy)methyl)-4-hydroxyphenoxy)benzylidene)amino)oxy)propanoic acid

¹H NMR:(DMSO-D₆, 400 MHz): 1.41(3H, d, J=6.8 Hz), 4.86-4.91(1H, q, J=6.8&7.2 Hz), 5.89 (1H, s), 6.50-6.53(1H, m), 6.68(1H, d, J=8.8 Hz), 6.87(1H, d, J=3.2 Hz), 7.23-7.29(4H, m), 7.96 (2H, s), 8.31(1H, s). % Yield: 20%

### EXAMPLE 35

### 2-(((3,5-dibromo-4-(3-((3 -chlorophenyl)(hydroxy)methyl)-4-hydroxyphenoxy)benzylidene)amino)oxy)acetic acid

¹H NMR:(DMSO-D₆, 400 MHz): 4.66(2H, s), 5.85(1H, m), 6.50-6.53(1H, dd, J=3.2&8.8 Hz), 6.68(1H, d, J=8.4 Hz), 6.87(1H, d, J=3.2 Hz), 7.21-7.31(4H, m), 7.97(2H, s), 8.32(1H, s). % Yield: 49%

### EXAMPLE 36

### 2-(((3,5-dibromo-4-(4-hydroxy-3-(piperidin-1-yl sulfonyl)phenoxy)benzylidene)amino)oxy)propanoic acid

### Step 1 Ethyl 2-(((3,5-dibromo-4-(3-(chlorosulfonyl)-4-methoxy phenoxy)benzylidene)amino)oxy)propanoate

Chlorosulfonic acid (0.99 g) was added to ethyl 2-(((3,5-dibromo-4-(4-methoxy phenoxy)benzylidene)amino)oxy)propanoate (Example 26, step 3) (0.89 g, 1.59 mmol) at 0-10° C. The reaction was stirred at 25° C. for 1 hr. Reaction mixture was poured in to ice-H₂O and taken up in ethyl acetate, washed with water, brine, dried over sodium sulphate to afford ethyl 2-(((3,5-dibromo-4-(3-(chlorosulfonyl)-4-methoxy phenoxy)benzylidene)amino)oxy)propanoate (1 g, 100% yield)

### Step 2. ethyl 2-(((3,5-dibromo-4-(4-methoxy-3-(piperidin-1-yl sulfonyl)phenoxy)benzylidene)amino)oxy)propanoate

To a solution of ethyl 2-(((3,5-dibromo-4-(3-(chlorosulfonyl)-4-methoxy phenoxy)benzylidene)amino)oxy)propanoate (0.41 g, 0.68 mmol) in dichloromethane (4.0 mL) was added piperidine (0.11 g, 1.36 mmol) at 0-10° C. followed by triethyl amine (0.138 g, 1.36 mmol). The reaction mixture was stirred at 20-25° C. for 2 hrs. The product was taken up in ethyl acetate, washed with water, brine, dried over sodium sulphate, filtered and concentrated to give the crude product, which was purified by column chromatography over flash silica gel (hexane:ethyl acetate 90:10) to afford pure ethyl 24(3,5-dibromo-4-(4-methoxy-3-(piperidin-1-yl sulfonyl)phenoxy)benzylidene)amino)oxy)propanoate (0.2 g, 45% yield)

¹H NMR:(CDCl₃, 400 MHz): 1.22-1.26(3H, m), 1.33-1.52(9H, m), 3.10-3.13(4H, m), 3.81(3H, s), 4.16-4.21(2H, q, J=6.8 & 7.2 Hz), 4.73-4.78(1H, q, J=6.8 & 7.2 Hz), 6.87(1H, d, 6.90-6.93(1H, dd, J=2.8&8.8 Hz), 7.26 (1H, d, J=2.8 Hz), 7.73(2H, s), 8.00 (1H, s)

### Step 3. 2-(((3,5-dibromo-4-(4-hydroxy-3-(piperidin-1-ylsulfonyl)phenoxy)benzylidene)amino)oxy)propanoic acid

A solution of ethyl 2-(((3,5-dibromo-4-(4-methoxy-3-(piperidin-1-yl sulfonyl)phenoxy)benzylidene)amino)oxy)propanoate (0.18 g, 0.277 mmol) in dichloromethane (1.8 mL) was cooled to −60 to −70° C. under N₂ atomsphere. To that 1M BBr₃ solution in dicloromethane (1.18 mL) was added dropwise. The reaction mixture was allowed to warm up to −20° C. over 4 h. then diluted with more CH₂Cl₂ (25 mL) and quenched with H₂O. After stirring at 20-25° C. for 10 min, organic phase was separated, washed with water, brine, dried over sodium sulphate, filtered and concentrated to give crude product. The crude product was purified by column chromatography over flash silica gel (Chloroform:Methanol 95:05) to give pure 2-(((3,5-dibromo-4-(4-hydroxy-3-(piperidin-1 -ylsulfonyl)phenoxy)benzylidene)amino)oxy)propanoic acid (0.12 g, 68% Yield)

¹H NMR:(CD₃OD, 400 MHz): 1.49-1.52(6H, m), 1.57(3H, m), 3.10-3.13(4H, m), 4.87(1H, m), 6.94(1H, d, J=2.8 Hz), 6.98(1H, d, J=8.8 Hz), 7.03(1H, d, J=3.2 Hz), 7.96(2H, s), 8.17(1H, s).

Using appropriate starting materials and suitable modifications of one or more of the processes described above (Example 36) either alone or in suitable combination of the steps disclosed therein, including suitable addition and/or deletion of steps as may be necessary, well within the scope of a person skilled in the art, the following compounds (Examples 37-42) were prepared in an analogous manner

### EXAMPLE 37

### 2-(((3,5-dibromo-4-(4-hydroxy-3-(N-isopropylsulfamoyl)phenoxy)benzylidene)amino)oxy)propanoic acid

¹H NMR: (CD₃OD, 400 MHz): 1.03(6H, d, J=6.8 Hz), 1.51(3H, d, J=7.2 Hz), 3.31-3.34(1H, m), 4.80-4.82(1H, m), 6.95(1H, d, J=8.8 Hz), 7.00-7.03(2H, m), 7.95(2H, s), 8.17(1H, s). % Yield: 50%

### EXAMPLE 38

### 2-(((3,5-dibromo-4-(3-(N,N-diethylsulfamoyl)-4-hydroxyphenoxy)benzylidene)amino)oxy)propanoic acid

¹H NMR: (CD₃OD, 400 MHz): 1.07(6H, t, J=7.2 Hz), 1.51(3H, d, J=6.8 Hz), 3.28-3.34(4H, m), 4.79-4.82(1H, m), 6.93(1H, d, J=8.8 Hz), 6.99(1H, d, J=3.2 Hz), 7.01-7.03(1H, m), 7.95(2H, s), 8.17(1H, s). % Yield: 54%

### EXAMPLE 39

### 2-(((3,5-dibromo-4-(3-(N-cyclohexylsulfamoyl)-4-hydroxyphenoxy)benzylidene)amino)oxy)propanoic acid

¹H NMR: (CDCl₃, 400 MHz): 1.11-1.22(6H, m), 1.51-1.55(4H, m), 1.61(3H, d, J=7.2 Hz), 3.11-3.14(1H, m), 4.89-4.91(11-1, m), 6.93(1H, d, J=2.8 Hz), 7.00(1H, d, J=8.8 Hz), 7.05-7.08(1H, dd, J=3.2&9.2 Hz), 7.82(2H, s), 8.09(1H, s). % Yield: 57%

### EXAMPLE 40

### 2-(((4-(3-(N-((1R,2R,4S)-bicyclo[2.2.1]heptan-2-yl)sulfamoyl)-4-hydroxyphenoxy)-3,5-dibromobenzylidene)amino)oxy)propanoic acid

¹H NMR:(CDCl₃, 400 MHz): 1.25-1.27(3H, m), 1.41-1.48(4H,m), 1.60-1.67(4H,m), 2.06(1H, bs), 2.21(1H, bs), 3.12-3.13(1H, m), 4.87-4.92(1H, q, J=7.2 Hz), 6.92 (1H, d, J=2.8 Hz), 7.02 (1H, d, J=8.8 Hz), 7.06-7.09(1H, dd, J=2.8&9.2 Hz), 7.82(2H, s), 8.09(1H, s). % Yield: 12%

### EXAMPLE 41

### 2-(((3,5-dibromo-4-(4-hydroxy-3-(pyrrolidin-1-ylsulfonyl)phenoxy)benzylidene)amino)oxy)acetic acid

¹H NMR:(DMSO-D₆, 400 MHz): 1.72(4H, t, J=6.8 Hz), 3.22(4H, t, J=6.6 Hz), 4.55(2H, s), 6.94 (1H, d, J=2.0 Hz), 7.00-7.05(2H, m), 7.99(2H, s), 8.31(1H, s). % Yield: 45%

### EXAMPLE 42

### 2-(((3,5-dibromo-4-(4-hydroxy-3-(pyrrolidin-1-ylsulfonyl)phenoxy)benzylidene)amino)oxy)propanoic acid

¹H NMR:(CDCl₃, 400 MHz): 1.61(3H, d, J=7.2 Hz), 1.81(4H, t, J=3.4 Hz), 3.25(4H, t, J=6.8 Hz), 4.89-4.91(1H, q, J=7.2 Hz), 6.91(1H, d, J=3.2 Hz), 7.02(1H, d, J=9.2 Hz), 7.08(1H, dd, J=2.8 & 8.8 Hz), 7.82(2H, s), 8.09(1H, s). % Yield: 52%

For the synthesis of the above mentioned compounds following intermediates were prepared.

### Intermediate 1

### Ethyl 2-(((3,5-dichloro-4-(3-isopropyl-4-methoxyphenoxy)benzylidene)amino)oxy)acetate

¹H NMR: (CDCl₃, 400 MHz): 1.18(6H, d, J=6.8 Hz), 1.27-1.29(3H, m),3.24-3.31(1H, m), 3.78 (3H, s), 4.24-4.29(2H, m), 4.72(2H, s), 6.43-6.46(1H, dd, J=3.2&8.8 Hz), 6.70(1H, d, J=9.2 Hz), 6.83(1H, d, J=3.2 Hz), 7.61(2H, s), 8.12(1H, s).

### Intermediate 2

### Ethyl 2-(((4-(3-(sec-butyl)-4-methoxyphenoxy)-3,5-dichlorobenzylidene)amino)oxy)acetate

¹H NMR: (CDCl₃, 400 MHz): 0.87-0.89(3H, m), 1.15(3H,d, J=7.2 Hz), 1.28-1.33(3H, m), 1.46-1.61(2H, m), 3.03-3.08(1H, m), 3.77(314, s), 4.23-4.29(2H, m), 4.72(2H, s), 6.47-6.50(1H, dd, J=3.2&9.2 Hz), 6.72(1H, d, J=8.8 Hz), 6.75-6.76(1H, m), 7.61(2H, s), 8.12(1H, s).

### Intermediate 3

### Ethyl 2-(((3,5-dibromo-4-(3-isopropyl-4-methoxyphenoxy)benzylidene)amino)oxy)acetate

¹H NMR: (CDCl₃, 400 MHz): 1.18(6H, d, J=6.8 Hz), 1.31(3H, t, J=7.8 Hz), 3.26-3.29(1H, m), 3.78(3H, s), 4.24-4.30(2H, q, J=7.2 Hz), 4.73(2H, s), 6.41-6.44(1H, dd, J=2.8&8.8 Hz), 6.70(1H, d, J=8.8 Hz), 6.82(1H, d, J=3.2 Hz), 7.81(2H, s), 8.11(1H, s).

### Intermediate 4

### Ethyl 2-(((3,5-dichloro-4-((6-methoxy-[1,1’-biphenyl]-3-yl)oxy)benzylidene)amino)oxy)acetate

¹H NMR: (CDCl₃, 400 MHz): 1.25-1.32(3H, m), 3.76(3H, s), 4.24-4.29(2H, m), 4.72(2H, s), 6.75-6.78(1H, dd, J=3.2&9.2 Hz), 6.82(1H, d, J=3.2 Hz), 6.89(1H, d, J=8.8 Hz), 7.31(1H, t, J=7.2 Hz), 7.39(2H, t, 7.49(2H, d, J=7.2 Hz), 7.61(2H, s), 8.11(1H, s).

### Intermediate 5

### Ethyl 2-(((4-(3-benzyl-4-methoxyphenoxy)-3,5-dichlorobenzylidene)amino)oxy)acetate

¹H NMR: (CDCl₃, 400 MHz): 1.27-1.33(3H, m), 3.7(3H, s), 3.94(2H, s), 4.24-4.29(2H, q, J=6.8 Hz), 4.72(2H, s), 6.52-6.55(1H, dd, J=3.2&8.8 Hz), 6.67(1H, d, J=3.2 Hz), 6.73(1H, d, J=8.8 Hz), 7.17-7.19(3H, m), 7.24-7.27(2H, m), 7.58(2H, s), 8.10(1H, s).

### Intermediate 6

### Ethyl 2-(((3,5-dibromo-4-(3-(sec-butyl)-4-methoxyphenoxy)benzylidene)amino)oxy)acetate

¹H NMR:(CDCl₃, 400 MHz): 0.83(3H, t, J=7.2 Hz), 1.15(3H, d, J=6.8 Hz), 1.30(3H, t, J=6.8 Hz), 1.31(2H, q, J=7.2 Hz), 3.03-3.09(1H, m), 3.77(3H, s), 4.26(2H, q, J=6. Hz), 4.72(2H, s), 6.47(1H, m), 6.72(2H, d, J=8.8 Hz), 7.81(2H, s), 8.1(1H, s).

### Intermediate 7

### Ethyl 2-(((3,5-dibromo-4-((6-methoxy-[1,1′-biphenyl]-3-yl)oxy)benzylidene)amino)oxy)acetate

¹H NMR:(CDCl₃, 400 MHz): 1.25-1.32(3H, m), 3.77(3H, s), 4.23-4.29(2H, m), 4.72(2H, s), 6.73-6.76(1H, dd, J=3.2&9.6 Hz), 6.81(1H, d, J=3.21 Hz), 6.90(1H, d, J=9.2 Hz), 7.30-7.31(1H, m), 7.37-7.41(2H, m), 7.50-7.52(2H, m), 7.82(2H, s), 8.11(1H, s).

### Intermediate 8

### Ethyl 2-(((3,5-dichloro-4-(3-isopropyl-4-methoxyphenoxy)benzylidene)amino)oxy)propanoate

¹H NMR: (CDCl₃, 400 MHz): 1.18(6H, d, J=7.2 Hz), 1.29(3H, d, J=7.2 Hz), 1.54(3H, d, J=7.2 Hz), 3.24-3.31(1H, m), 3.78(3H, s), 4.23-4.28(2H, m), 4.79-4.85(1H, q, J=7.2 Hz), 6.43-6.46(1H, dd, J=2.8&8.8 Hz), 6.69(1H, d, J=8.8 Hz), 6.84(1H, d, J=3.2 Hz), 7.59(2H, s), 8.08(1H, s).

### Intermediate 9

### Ethyl 2-(((4-(3-benzyl-4-methoxyphenoxy)-3,5-dibromobenzylidene)amino)oxy)acetate

¹H NMR: (CDCl₃, 400 MHz): 1.27-1.33(3H, m), 3.76(3H, s), 3.93(2H, s), 4.24-4.29(2H, q, J=6.8 & 7.2 Hz), 4.73(2H, s) 6.51-6.54(1H, dd, J=3.2&8.8 Hz), 6.65(1H, d, J=3.21 Hz), 6.73(1H, d, J=8.8 Hz), 7.15-7.19(3H, m), 7.24-7.29(2H, m), 7.79(2H, s), 8.10(1H, s).

### Intermediate 10

### Ethyl 2-(((3,5-dibromo-4-(3-isopropyl-4-methoxyphenoxy)benzylidene)amino)oxy)propanoate

¹H NMR: (CDCl₃, 400 MHz): 1.18(6H, d, J=6.8 Hz), 1.29(3H, t, J=7.2 Hz), 1.56(3H, d, J=7.2 Hz), 3.24-3.31(1H, m), 3.78(3H, s), 4.22-4.29(2H, m), 4.80-4.85(1H, q, J=7.2 Hz), 6.41-6.44(1H, dd, J=2.8&8.8 Hz),6.70(1H, d, J=8.8 Hz), 7.82(1H, d, J=2.8 Hz), 7.80(2H, s), 8.07(1H, s).

### Intermediate 11

### Ethyl 2-(((3,5-dibromo-4-((6-methoxy-[1,1′-biphenyl]-3-yl)oxy)benzylidene)amino)oxy)propanoate

¹H NMR: (CDCl₃, 400 MHz): 1.30(3H, t, J=7.0 Hz), 1.54(3H, d, J=6.8 Hz), 3.77(3H, s), 4.23-4.28 (2H, q, J=6.8&7.2 Hz), 4.79-4.84(1H, q, J=6.8&7.2 Hz), 6.73-6.76(1H, dd, J=3.2&9.2 Hz), 6.81(1H, d, J=2.8 Hz), 6.89(1H, d, J=8.8 Hz), 7.29-7.33(1H, m), 7.39(2H, t, J=7.2.0 Hz), 7.51(2H, t, J=7.2 Hz), 7.80(2H, s), 8.06(1H, s).

### Intermediate 12

### Ethyl 2-(((3,5-dibromo-4-(3-isopropyl-4-methoxyphenoxy)benzylidene)amino)oxy)-2-methyl propanoate

¹H NMR: (CDCl₃, 400 MHz): 1.22(6H, d, J=7.2 Hz), 1.31(3H, t, J=7 Hz), 1.56(3H, s), 1.58(3H, s), 3.13-3.20(1H, m), 3.96(3H, s), 4.21-4.27(2H, q, J=7 Hz), 6.41-6.45(1H, dd, J=3.2&8.81 Hz), 6.69(1H, d, J=8.4 Hz), 6.99(1H, d, J=3.2 Hz), 7.77(2H, s), 8.02(1H, s).

### Intermediate 13

### Ethyl 2-(((3,5-dibromo-4-(3-isopropyl-4-methoxyphenoxy)benzylidene)amino)oxy)butanoate

¹H NMR: (CDCl₃, 400 MHz): 1.05(3H, t, J=7.2 Hz), 1.18(6H, d, J=6.8 Hz), 1.30(3H, t, J=7.2 Hz), 1.89-1.97(2H, m), 3.24-3.31(1H, m), 3.78(3H, s), 4.21-4.29(2H, m), 4.66-4.69(1H, q, J=5.6& 7.2 Hz), 6.41-6.44 (1H, dd, J=3.2&8.8 Hz), 6.70(1H, d, J=8.8 Hz), 6.82(1H, d, J=2.8 Hz), 7.80(2H, s), 8.09(1H, s).

### Intermediate 14

### Ethyl 2-(((3,5-dibromo-4-(3-(sec-butyl)-4-methoxyphenoxy)benzylidene)amino)oxy)propanoate

¹H NMR: (CDCl₃, 400 MHz): 0.83(3H, t, J=7.2 Hz), 1.15(3H, d, J=6.8 Hz), 1.30(3H, t, J=7.2 Hz), 1.52-1.55(2H, m), 1.57(3H, d, J=7.2 Hz), 3.03-3.09(1H, m), 3.77(3H, s), 4.23-4.28(2H, q, J=7.2 Hz), 4.79-4.85(1H, q, J=7.2 Hz), 6.45-6.48(1H, dd, J=3.2 Hz&8.8 Hz), 6.71(1H, d, J=8.8 Hz), 6.74 (1H, d, J=3.2 Hz), 7.79(2H, s), 8.07(1H, s).

### Intermediate 15

### Ethyl 2-(((3,5-dibromo-4-(3-(sec-butyl)-4-methoxyphenoxy)benzylidene)amino)oxy)-2-methylpropanoate

¹H NMR: (CDCl₃, 400 MHz): 0.83(314, t, J=7.4 Hz), 1.15(3H, d, J=6.8 Hz), 1.26-1.3(3H, m), 1.47-1.56(2H, m), 1.58(6H, s), 3.04-3.09(1H, m), 3.77(3H, s), 4.21-4.26(2H, q, J=6.8 Hz &7.2 Hz), 6.44-6.47(1H, dd, J=3.2&8.8 Hz), 6.71(1H, d, J=8.8 Hz), 6.75(1H, d, J=3.2 Hz), 7.77(2H, s), 8.01 (1H, s).

### Intermediate 16

### Ethyl 2-(((4-(3-benzyl-4-methoxyphenoxy)-3,5-dibromobenzylidene)amino)oxy)propanoate

¹H NMR:(CDCl₃, 400 MHz): 1.27(314, t, J=7.2 Hz), 1.54(3H, d, J=6.8 Hz), 3.75(3H, s), 3.92(2H, s), 4.22-4.27(2H, q, J=7.2 Hz), 4.79-4.84(1H, q, J=7.2 Hz), 6.51-6.54(1H, dd, J=3.2&8.8 Hz), 6.65(1H, d, J=3.2 Hz), 6.73(1H, d, J=8.8 Hz), 7.12-7.27(5H, m), 7.77(2H, s), 8.08(1H, s).

### Intermediate 17

### Ethyl 2-(((4-(3-benzyl-4-methoxyphenoxy)-3,5-dibromobenzylidene)amino)oxy)butanoate

¹H NMR:(CDCl₃, 400 MHz): 1.05(3H, t, J=7.2 Hz), 1.30(3H, t, J=7.2 Hz), 1.90-196(2H, m), 3.75(3H, s), 3.92(2H, s), 4.25(2H, q, J=7.2 Hz), 4.67(1H, t, J=5.6 Hz), 6.51-6.54(1H, dd, J=3.2 & 8.8 Hz), 6.65(1H, d, J=3.2 Hz), 6.73(1H, d, J=8.8 Hz), 7.17-7.27(5H, m), 7.77(2H, s), 8.08(1H, s).

### Intermediate 18

### Ethyl 2-(((3,5-dibromo-4-(3-(sec-butyl)-4-methoxyphenoxy)benzylidene)amino)oxy)-2-phenyl acetate

¹H NMR:(CDCl₃, 400 MHz): 0.83(3H, t, J=7.4 Hz), 1.15(3H, d, J=6.8 Hz), 1.26(3H, t, J=7.0 Hz), 1.46-1.60(2H, m), 3.04-3.09(1H, m), 3.77(3H, s), 4.18-4.32(2H, m), 5.70(1H, s), 6.45-6.48 (1H, dd, J=3.2&8.8 Hz), 6.70-6.74(2H, m), 7.39-7.5(3H, m), 7.5-7.53(2H, m), 7.81(2H, s), 8.2(1H, s).

### Intermediate 19

### Ethyl 2-(((3,5-dibromo-4-(3-(sec-butyl)-4-ethoxy phenoxy)benzylidene)amino)oxy)butanoate

¹H NMR:(CDCl₃, 400 MHz): 0.85-0.92(3H, m), 1.06(3H, t, J=7.2 Hz), 1.14(3H, d, J=7.2 Hz), 1.30(3H, d, J=6.8 Hz), 1.44-1.61-(2H, m), 1.89-1.99(2H, m), 3.03-3.09(1H, m), 3.77(3H, s), 4.21-4.29(2H, m), 4.66-4.69(1H, m), 6.45-6.48(1H, dd, J=2.8&8.8 Hz), 6.72(1H, d, J=9.2 Hz), 6.74 (1H, d, J=3.2 Hz), 7.79(2H, s), 8.09(1H, s).

### Intermediate 20

### Ethyl 2-(((3,5-dibromo-4-((6-methoxy-[1,1′-biphenyl]-3-yl)oxy)benzylidene)amino)oxy)-2-methylpropanoate

¹H NMR:(CDCl₃, 400 MHz): 1.26(3H, t, J=7.2 Hz), 1.58(6H, s), 3.76(3H, s), 4.20-4.26(2H, q, J=6.8 &7.2 Hz), 6.73-6.76(1H, dd, J=2.8&8.8 Hz), 6.81(1H, d, J=3.2 Hz), 6.89(1H, d, J=8.8 Hz), 7.29-7.33(1H, m), 7.37-7.40(2H, m), 7.48-7.51(2H, m), 7.77(2H, s), 8.00(1H, s).

### Intermediate 21

### Ethyl 2-(((3,5-dibromo-4-((6-methoxy-[1,1′-biphenyl]-3-yl)oxy)benzylidene)amino)oxy)butanoate

¹H NMR:(CDCl₃, 400 MHz): 1.05(3H, t, J=7.2 Hz), 1.30(3H, t, J=7.2 Hz), 1.88-1.95(2H, m), 3.76(3H, s), 4.22-4.28(2H, m), 4.67(1H, t, J=5.6 Hz), 6.74(1H, dd, J=3.2&8.8 Hz), 6.8(1H, d, J=3.2 Hz), 6.89(1H, d, J=9.2 Hz), 7.29-7.33(1H, m), 7.37-7.41(2H, m), 7.49-7.52(2H, m), 7.8(2H, s), 8.08(1H,s).

### Intermediate 22

### Ethyl 2-(((4-(3-benzyl-4-methoxyphenoxy)-3,5-dibromobenzylidene)amino)oxy)-2-methyl propanoate

¹H NMR: (CDCl₃, 400 MHz): 1.28(3H, t, J=7.2 Hz), 1.55(3H, s), 1.58(3H, s), 3.75(3H, s), 3.92 (2H, s), 4.21-4.26(2H, q, J=7.2 Hz), 6.51-6.54(1H, dd, J=3.2&8.8 Hz), 6.65(1H, d, J=2.8 Hz), 6.73 (1H, d, J=9.2 Hz), 7.12-7.19(3H, m), 7.23-7.27(2H, m), 7.75(2H, s), 8.00(1H, s).

### Intermediate 23

### Ethyl 2-(((4-(3-(sec-butyl)-4-methoxyphenoxy)-3,5-dichlorobenzylidene)amino)oxy)propanoate

¹H NMR: (CDCl₃, 400 MHz): 0.83(3H, t, J=7.6 Hz), 1.15(3H, d, J=6.8 Hz), 1.30(3H, t, J=7.2 Hz), 1.55-1.61(5H, m), 3.03-3.08(1H, m), 3.77(3H, s), 4.22-4.28(2H, m), 4.79-4.85(1H, q, J=6.8& 7.2 Hz), 6.46-6.49(1H, dd, J=3.2 Hz&8.8 Hz), 6.72(1H, d, J=8.8 Hz), 6.76(1H, d, J=3.2 Hz), 7.59(2H, s), 8.08(1H, s).

### Intermediate 24

### Ethyl 2-(((3,5-dibromo-4-(3-(tert-butyl)-4-methoxyphenoxy)benzylidene)amino)oxy)propanoate

¹H NMR: (CDCl₃, 400 MHz): 1.28(3H, t, J=7.2 Hz), 1.34(9H, s), 1.58(3H, d, J=6.8 Hz), 3.78(3H, s), 4.22-4.28(2H, q, J=6.8&7.2 Hz), 4.79-4.84(1H, q, J=6.8&7.2 Hz), 6.40-6.43(1H, dd, J=3.2& 8.8 Hz), 6.71(1H, d, J=8.8 Hz), 6.93(1H, d, J=3.2 Hz), 7.79(2H, s), 8.06(1H, s).

### Intermediate 25

### Ethyl 2-(((3,5-dibromo-4-(3-ethyl-4-methoxyphenoxy)benzylidene)amino)oxy)propanoate

¹H NMR: (CDCl₃, 400 MHz): 1.15-1.18(3H, t, J=7.2 Hz), 1.27-1.29(3H, t, J=7.2 Hz), 1.56(3H, d, J=8.4 Hz), 2.57-2.62(2H, q, J=7.2&7.6 Hz), 3.78(3H, s), 4.23-4.28(2H, q, J=7.2 Hz), 4.8-4.85(1H, q, J=6.8&7.2 Hz), 6.47-6.50(1H, dd, J=3.2&8.8 Hz), 6.69-6.72(2H, m), 7.80(2H, s), 8.07(1H, s).

### Intermediate 26

### Ethyl 2-(((3,5-dibromo-4-(3-(4-chlorobenzoyl)-4-methoxyphenoxy)benzylidene)amino)oxy)propanoate

¹H NMR: (CDCl₃, 400 MHz): 1.22-1.27 (3H, m), 1.45-1.58(3H, m), 3.69(3H, s), 4.23-4.28(2H, m), 4.81-4.87(1H, m), 6.82(1H, d, J=2.8 Hz), 6.91-6.98(2H, m), 7.24(1H, m), 7.39-7.42(2H, d, J=8.4 Hz), 7.73-7.77(3H, m), 8.19(1H, s).

### Intermediate 27

### Ethyl 2-(((3,5-dibromo-4-(3-(4-chlorobenzoyl)-4-hydroxyphenoxy)benzylidene)amino)oxy)propanoate

¹H NMR:(CDCl₃, 400 MHz): 1.22-1.27(3H, m), 1.45-1.58(3H, m), 4.23-4.28(2H, m), 4.81-4.87 (1H, m), 6.97-7.06(3H, m), 7.44(2H, d, J=8.8 Hz), 7.63(2H, d, J=8.4 Hz), 7.77(2H, s), 8.19 (1H, s).

### Intermediate 28

### Ethyl 2-(((3,5-dibromo-4-(3-(4-chlorobenzoyl)-4-hydroxyphenoxy)benzylidene)amino)oxy)acetate

¹H NMR: (CDCl₃, 400 MHz): 1.31(3H, t, J=7.2 Hz), 4.24-4.27(2H, q, J=7.2 Hz), 4.72(2H, s), 6.97 (1H, d, J=2.8 Hz), 7.03-7.09(2H, m), 7.45(2H, d, J=8.4 Hz), 7.64(2H, d, J=6.8 Hz), 7.79(2H, s), 8.09(1H, s).

### Intermediate 29

### Ethyl 2-(((3,5-dibromo-4-(3-(3-chlorobenzoyl)-4-hydroxyphenoxy)benzylidene)amino)oxy)propanoate

¹H NMR:(CDCl₃, 400 MHz): 1.22-1.27(3H, m), 1.45-1.58(3H, m), 4.24-4.29(2H, q, J=7.2 Hz), 4.80-4.85 (1H, q, J=6.8 Hz), 6.86(1H, d, J=2.8 Hz), 7.08(1H, d, J=9.2 Hz), 7.17-7.20(1H, dd, J=3.2 &9.2 Hz), 7.40(1H, t, J=8.0 Hz), 7.53-7.63(3H, m), 7.78(2H, s), 8.05(1H, s).

### Intermediate 30

### Ethyl 2-(((3,5-dibromo-4-(3-(4-bromobenzoyl)-4-hydroxyphenoxy)benzylidene)amino)oxy)acetate

¹H NMR:(CDCl₃, 400 MHz): 1.31(3H, t, J=7.2 Hz), 4.24-4.27(2H, q, J=7.2 Hz), 4.72(2H, s), 6.97 (1H, d, J=2.8 Hz), 7.04-7.08(2H, m), 7.55(2H, d, J=8.4 Hz), 7.62(2H, d, J=8.4 Hz), 7.79(2H, s), 8.09(1H, s).

### Intermediate 31

### Ethyl 2-(((3,5-dibromo-4-(3-(3-chlorobenzoyl)-4-hydroxyphenoxy)benzylidene)amino)oxy)acetate

¹H NMR: (CDCl₃, 400 MHz): 1.31(3H, t, J=7.2 Hz), 4.24-4.29(2H, q, J=7.2 Hz), 4.72(2H, s), 6.87 (1H, d, J=3.2 Hz), 7.06(1H, d, J=9.2 Hz), 7.16-7.19(1H, dd, J=2.8&9.2 Hz), 7.39(1H, t, J=7.8 Hz), 7.52-7.61(3H, m), 7.79(2H, s), 8.08(1H, s).

### Intermediate 32

### Ethyl 2-(((3,5-dibromo-4-(4-methoxy-3-(piperidin-1-ylsulfonyl)phenoxy)benzylidene)amino)oxy)propanoate

¹H NMR: (CDCl₃, 400 MHz): 1.22-1.26(3H, m), 1.33-1.52(91-1, m), 3.10-3.13(4H, m), 3.81(3H, s), 4.16-4.21(2H, q, J=6.8&7.2 Hz), 4.73-4.78(1H, q, J=6.8 &7.2 Hz), 6.87(1H, d, J=8.8 Hz), 6.90-6.93(1H, dd, J=2.8&8.8 Hz), 7.26(1H, d, J=2.8 Hz), 7.73 (2H, s), 8.00(1H, s)

### Intermediate 33

### Ethyl 2-(((3,5-dibromo-4-(3-(N-isopropylsulfamoyl)-4-methoxyphenoxy)benzylidene)amino)oxy)propanoate

¹H NMR: (CDCl₃, 400 MHz): 1.05(6H, d, J=6.8 Hz), 1.29-1.33(3H, t, J=7.2 Hz), 1.56(3H, d, J=5.6 Hz), 3.40-3.45(1H, m), 3.95(3H, s), 4.23-4.28(2H, q, J=6.8&7.2 Hz), 4.74-4.85(1H, q, J=6.8 &7.2 Hz), 6.95-7.03(2H, m), 7.36(1H, d, J=3.2 Hz), 7.80(2H, s), 8.20(1H, s).

### Intermediate 34

### Ethyl 2-(((3,5-dibromo-4-(3-(N,N-diethylsulfamoyl)-4-methoxyphenoxy)benzylidene)amino)oxy)propanoate

¹H NMR: (CDCl₃, 400 MHz): 1.09(6H, t, J=7.2 Hz), 1.29-1.32(3H, t, J=7.2 Hz), 1.56-1.51(3H, m), 3.30-3.36(4H, q, J=7.2 Hz), 3.89(3H, s), 4.23-4.28(2H, q, J=7.2 Hz), 4.80-4.85(1H, q, J=6.8&7.2 Hz), 6.90-7.01(2H, m), 7.38(1H, d, J=2.8 Hz), 7.80(2H, s), 8.07(1H, s).

### Intermediate 35

### Ethyl 2-(((3,5-dibromo-4-(3-(N-cyclohexylsulfamoyl)-4-methoxyphenoxy)benzylidene)amino)oxy)propanoate

¹H NMR: (CDCl₃, 400 MHz): 1.09-1.16(6H, m), 1.20-1.25(4H, m), 1.31(3H, t, J=7.0 Hz), 1.55 (3H, d, J=6.8 Hz), 2.04-3.13(1H, m), 3.95(3H, s), 4.23-4.26(2H, q, J=7.2 Hz), 4.81-4.85(1H, m), 6.97(1H, d, J=8.8 Hz), 7.01-7.03(1H, dd, J=3.2&9.3 Hz), 7.33(1H, d, J=2.8 Hz), 7.80(2H, s), 8.07 (1H, s).

### Intermediate 36

### Ethyl 2-(((4-(3-(N-((1R,2R,4S)-bicyclo[2.2.1]heptan-2-yl)sulfamoyl)-4-methoxyphenoxy)-3,5-dibromobenzylidene)amino)oxy)propanoate

¹H NMR: (CDCl₃, 400 MHz): 0.98-0.99 (3H, m), 1.10-1.18(3H, m), 1.28-1.33(6H, m), 1.37-1.41 (2H, m), 2.02-2.04(2H, m), 3.09-3.13(1H, m), 3.96(3H, s), 4.23-4.28(2H, q, J=7.2 Hz), 4.80-4.86(1H, q, J=6.8 Hz), 6.97(1H, d, J=9.2 Hz), 7.02-7.05(1H, dd, J=3.2&9.2 Hz), 7.34 (1H, d, J=3.2 Hz), 7.80(2H, s), 8.07(1H, s).

### Intermediate 37

### Ethyl 2-(((3,5-dibromo-4-(4-methoxy-3-(pyrrolidin-1-ylsulfonyl)phenoxy)benzylidene)amino)oxy)acetate

¹H NMR: (CDCl₃, 400 MHz): 1.31(3H, t, J=7.2 Hz), 1.83(4H, t, J=3.6 Hz), 3.37(4H, t, J=7 Hz), 3.90(3H, s), 4.24-4.30(2H, q, J=7.2 Hz), 4.73(2H, s), 6.96-6.97(2H, m), 6.39(1H, d, J=2.8 Hz), 7.82(2H, s), 8.11(1H, s).

### Intermediate 38

### Ethyl 2-(((3,5-dibromo-4-(4-methoxy-3-(pyrrolidin-1-ylsulfonyl)phenoxy)benzylidene)amino)oxy)propanoate

¹H NMR: (CDCl₃, 400 MHz): 1.31(3H, t, J=7.0 Hz), 1.55(3H, d, J=6.8 Hz), 1.82-1.84(4H, m), 3.37(4H, t, J=6.6 Hz), 3.90(3H, s), 4.23-4.28(2H, q, J=7.2 Hz), 4.08-4.85(1H, q, J=7.2 Hz), 6.94(1H, d, J=9.2 Hz), 6.97-7.00(1H, dd, J=2.8&8.8 Hz), 7.38(1H, d, J=2.8 Hz), 7.80(2H, s), 8.06(1H, s).

### Intermediate 39

### 3,5-Dichloro-4-(3-isopropyl-4-methoxy-phenoxy)-benzaldehyde oxime

¹H NMR: (CDCl₃, 400 MHz): 1.18(6H, d, J=7.2 Hz), 3.24-3.31(1H, m), 3.78(3H, s), 6.44-6.47(1H, dd, J=2.8 & 8.8 Hz), 6.70(1H, d, J=8.8 Hz), 6.84(1H, d, J=2.8 Hz), 7.61(2H, s), 8.05(1H,s).

### Intermediate 40

### 4-(3-sec-Butyl-4-methoxy-phenoxy)-3,5-dichloro-benzaldehyde oxime

¹H NMR: (CDCl₃, 400 MHz): 0.83(3H, t, J=7.4 Hz), 1.15(3H, d, J=6.8 Hz),1.46-1.55(2H, m), 3.04-3.09(1H, m), 3.77(3H, s), 6.47-6.50(1H, dd, J=3.2&8.8 Hz), 6.72(1H, d, J=9.2 Hz), 6.77(1H, d, J=3.2 Hz), 7.61(2H, s), 8.04(1H, s).

### Intermediate 41

### 3,5-Dibromo-4-(3-isopropyl-4-methoxy-phenoxy)-benzaldehyde oxime

¹H NMR: (CDCl₃, 400 MHz): 1.18(6H, d, J=6.8 Hz), 3.26-3.30(1H, m), 3.78(3H, s), 6.42-6.45(1H, dd, J=2.8&8.8 Hz), 6.70(1H, d, J=8.8 Hz), 6.83(1H, d, J=3.2 Hz), 7.82(2H, s), 8.04(1H, s)

### Intermediate 42

### 4-(3-Benzyl-4-methoxy-phenoxy)-3,5-dichloro-benzaldehyde oxime

¹H NMR: (CDCl₃, 400 MHz): 3.75(3H, s), 3.93(2H, s), 6.54-6.56(1H, dd, J=2.8&8.8 Hz), 6.68(1H, d, J=3.2 Hz), 6.73(1H, d, J=9.2 Hz), 7.15-7.19(3H, m), 7.24-7.27(2H, m), 7.58(2H, s), 8.03(1H, s).

### Intermediate 43

### 3,5-Dibromo-4-(3-sec-butyl-4-methoxy-phenoxy)-benzaldehyde oxime

¹H NMR:(CDCl₃, 400 MHz): 0.83(3H, t, J=7.4 Hz), 1.15(3H, d, J=6.8 Hz), 1.46-1.61(2H, m), 3.04-3.09(1H, m), 3.77(3H, s), 6.45-6.48(1H, dd, J=3.2 Hz&8.8 Hz), 6.72(1H, d, J=8.8 Hz), 6.75(1H, d, J=3.2 Hz), 7.81(2H, s), 8.04(1H, s).

### Intermediate 44

### 4-(3-Benzyl-4-methoxy-phenoxy)-3,5-dibromo-benzaldehyde oxime

¹H NMR: (CDCl₃, 400 MHz): 3.75(3H, s), 3.92(2H, s), 6.52-6.55(1H, dd, J=3.2&8.8 Hz), 6.66(1H, d, J=2.8 Hz), 6.73(1H, d, J=8.8 Hz), 7.15-7.19(3H, m), 7.23-7.27(2H, m), 7.79(2H, s), 8.02(1H, s).

### Intermediate 45

### 3,5-Dibromo-4-(3-tert-butyl-4-hydroxy-phenoxy)-benzaldehyde oxime

¹H NMR:(CDCl₃ 400 MHz): 1.34(9H, s), 3.79(3H, s), 6.41-6.44(1H, dd, J=3.2&8.8 Hz), 6.71(1H, d, J=8.8 Hz), 6.94(1H, d, J=3.2 Hz), 7.81(2H, s), 8.04(1H, s).

### Intermediate 46

### 3,5-Dibromo-4-(3-ethyl-4-methoxy-phenoxy)-benzaldehyde oxime

¹H NMR: (CDCl₃, 400 MHz): 1.17(3H, t, J=7.6 Hz), 2.57-2.63(2H, q, J=7.6 Hz) 3.78(3H, s), 6.48-6.50(1H, dd, J=2.8 & 8.8 Hz), 6.71(1H, d, J=9.2 Hz), 6.73(1H, d, J=3.2 Hz), 7.82(2H, s), 8.04(1H, s).

### Intermediate 47

### 3,5-Dichloro-4-(3-isopropyl-4-methoxy-phenoxy)-benzaldehyde

¹H NMR: (CDCl₃, 400 MHz): 1.18(6H, d, J=6.8 Hz), 3.27-3.30(1H, m), 3.79(3H, s), 6.44-6.47(1H, dd, J=3.2&9.2 Hz), 6.70(1H, d, J=8.8 Hz), 6.85(1H, d, J=3.2 Hz), 7.91(2H, s), 9.93(1H, s).

### Intermediate 48

### 4-(3-sec-Butyl-4-methoxy-phenoxy)-3,5-dichloro-benzaldehyde

¹H NMR: (CDCl₃, 400 MHz): 0.88-0.89(3H, m), 1.15(3H, d, J=6.8 Hz), 1.47-1.61(2H, m), 3.03-3.11(1H, m), 3.78(3H, s), 6.47-6.50(1H, dd, J=3.2&8.8 Hz), 6.72(1H, d, J=8.8 Hz), 6.77(1H, d, J=2.8 Hz), 7.91 (2H, s), 9.93(1H, s).

### Intermediate 49

### 3,5-Dibromo-4-(3-isopropyl-4-methoxy-phenoxy)-benzaldehyde

¹H NMR: (CDCl₃, 400 MHz): 1.18(6H, d, J=6.8 Hz), 3.20-3.30(1H, m), 3.79(3H, s), 6.43-6.44(1H, dd, J=3.2&8.8 Hz), 6.70(1H, d, J=8.8 Hz), 6.83(1H, d, J=3.2 Hz), 8.10(2H, s), 9.92(1H, s).

### Intermediate 50

### 4-(3-Benzyl-4-methoxy-phenoxy)-3,5-dichloro-benzaldehyde

¹H NMR: (CDCl₃, 400 MHz): 3.76(3H, s), 3.92(2H, s), 6.54-6.57(1H, dd, J=3.2&8.8 Hz), 6.66(1H, d, J=3.2 Hz), 6.74(1H, d, J=9.2 Hz), 7.15-7.29(5H, m), 7.88(2H, s), 9.91(1H, s).

### Intermediate 51

### 3,5-Dibromo-4-(3-sec-butyl-4-methoxy-phenoxy)-benzaldehyde

¹H NMR: (CDCl₃, 400 MHz): 0.85(3H, t, J=7.4 Hz), 1.14(3H, d, J=6.8 Hz), 1.47-1.59(2H, m), 3.07-3.09(1H, m), 3.77(3H, s), 6.45-6.48(1H, dd, J=3.2&8.8 Hz), 6.72(1H, d, J=8.8 Hz), 6.75(1H, d, J=3.2 Hz), 8.11(2H, s), 9.90(1H, s).

### Intermediate 52

### 4-(3-Benzyl-4-methoxy-phenoxy)-3,5-dibromo-benzaldehyde

¹H NMR:(CDCl₃, 400 MHz): 3.76(3H, s), 3.93(2H, s), 6.52-6.55(1H, dd, J=2.8&8.8 Hz), 6.64(1H, d, J=3.2 Hz), 6.76(1H, d, J=8.8 Hz), 7.17-7.19(5H, m), 8.08(2H, s),9.91(1H, s).

### Intermediate 53

### 3,5-Dibromo-4-(3 -tert-butyl-4-methoxy-phenoxy)-benzaldehyde

¹H NMR:(CDCl₃, 400 MHz): 1.34(9H, s), 3.85(3H, s), 6.40-6.43(1H, dd, J=3.2 Hz & 8.8 Hz), 6.72(1H, d, J=8.8 Hz), 6.93(1H, d, J=3.2 Hz), 8.11(2H, s), 9.92(1H, s).

### Intermediate 54

### 3,5-Dibromo-4-(3-ethyl-4-methoxy-phenoxy)-benzaldehyde

¹H NMR:(CDCl₃, 400 MHz): 1.17(3H, t, J=7.6 Hz), 2.57-2.63(2H, q, J=7.2&7.6 Hz), 3.79(314, s), 6.48-6.51(1H, dd, J=3.2&8.8 Hz), 6.70-6.74(2H, m), 8.11(2H, s),9.92(1H, s).

### Activity Data:

In vitro TR-α & TR-β activities were determined as per in-house protocols and the results of representative compounds are provided in tables 1 & 2 below as a proof of the efficacies of the novel class of compounds disclosed above.

TABLE 1

| | EC₅₀ TR- | EC₅₀ TR- | EC₅₀ |
|---|---|---|---|
| Example No | α (nM) | β(nM) | TR α/β |
| 7 | 5.2 | 2.9 | 1.79 |
| 10 | 25 | 3.25 | 7.69 |
| 14 | 5.12 | 0.99 | 5.17 |
| 30 | 143 | 166 | 0.86 |
| 35 | 2100 | 4900 | 0.43 |

TABLE 2

| | Conc. | | | | Conc. | | |
|---|---|---|---|---|---|---|---|
| Ex. No | (nM) | TR-α * | TR-β * | Ex. No | (nM) | TR-α * | TR-β * |
| 1 | 1 | 11.78 | 7.72 | 13 | 1 | 29.62 | 23.09 |
| | 10 | 25.70 | 20.55 | | 10 | 63.87 | 73.39 |
| | 100 | 73.47 | 52.56 | | 100 | 76.83 | 86.70 |
| | 1000 | 98.95 | 53.36 | | 1000 | 56.92 | 94.56 |
| 2 | 1 | 10.17 | 13.96 | 15 | 1 | 35.68 | 23.03 |
| | 10 | 26.68 | 34.48 | | 10 | 77.66 | 84.29 |
| | 100 | 66.74 | 79.36 | | 100 | 118.9 | 118.4 |
| | 1000 | 107.7 | 115.1 | | 1000 | 122.8 | 72.35 |
| 3 | 1 | 18.28 | 17.67 | 16 | 1 | 19.08 | 12.02 |
| | 10 | 38.46 | 49.52 | | 10 | 29.42 | 31.73 |
| | 100 | 93.75 | 92.11 | | 100 | 119.35 | 54.32 |
| | 1000 | 133.17 | 138.18 | | 1000 | 160.99 | 70.46 |
| 4 | 1 | 10.97 | 11.40 | 17 | 1 | 21.62 | 21.20 |
| | 10 | 09.45 | 10.47 | | 10 | 38.45 | 31.32 |
| | 100 | 14.04 | 19.34 | | 100 | 66.20 | 81.54 |
| | 1000 | 35.52 | 64.37 | | 1000 | 109.0 | 85.64 |
| 5 | 1 | 14.22 | 14.76 | 18 | 1 | 15.53 | 13.75 |
| | 10 | 10.76 | 11.97 | | 10 | 36.02 | 20.83 |
| | 100 | 28.33 | 43.57 | | 100 | 76.97 | 69.45 |
| | 1000 | 56.98 | 80.45 | | 1000 | 90.41 | 122.2 |
| 6 | 1 | 11.61 | 15.93 | 19 | 1 | 34.38 | 20.25 |
| | 10 | 39.79 | 37.12 | | 10 | 80.77 | 69.74 |
| | 100 | 60.62 | 83.99 | | 100 | 64.21 | 86.23 |
| | 1000 | 64.94 | 74.58 | | 1000 | 108.9 | 90.81 |
| 8 | 1 | 12.37 | 16.26 | 20 | 1 | 9.45 | 8.98 |
| | 10 | 29.47 | 45.43 | | 10 | 13.11 | 14.84 |
| | 100 | 74.98 | 85.16 | | 100 | 34.46 | 45.96 |
| | 1000 | 76.64 | 89.04 | | 1000 | 74.27 | 98.61 |
| 9 | 1 | 10.74 | 11.50 | 21 | 1 | 12.79 | 12.94 |
| | 10 | 10.73 | 17.59 | | 10 | 16.59 | 18.17 |
| | 100 | 23.77 | 60.18 | | 100 | 50.37 | 51.58 |
| | 1000 | 64.81 | 98.76 | | 1000 | 104.6 | 63.76 |
| 11 | 1 | 16.24 | 16.07 | 22 | 1 | 12.07 | 9.09 |
| | 10 | 32.78 | 39.28 | | 10 | 27.23 | 24.91 |
| | 100 | 70.18 | 73.81 | | 100 | 48.60 | 44.92 |
| | 1000 | 49.40 | 65.31 | | 1000 | 85.07 | 73.91 |
| 12 | 1 | 23.98 | 39.04 | 23 | 1 | 22.59 | 19.22 |
| | 10 | 59.93 | 65.38 | | 10 | 48.23 | 53.13 |
| | 100 | 88.71 | 119.15 | | 100 | 92.19 | 78.93 |
| | 1000 | 110.33 | 67.44 | | 1000 | 137.5 | 90.01 |
| 24 | 1 | 18.53 | 16.61 | 34 | 1 | 14.07 | 10.89 |
| | 10 | 52.10 | 48.85 | | 10 | 11.75 | 9.74 |
| | 100 | 101.79 | 105.07 | | 100 | 17.86 | 21.45 |
| | 1000 | 120.92 | 86.08 | | 1000 | 39.84 | 47.46 |
| 25 | 1 | 13.84 | 11.51 | 36 | 1 | 14.14 | 10.98 |
| | 10 | 28.54 | 37.08 | | 10 | 15.36 | 19.66 |
| | 100 | 79.04 | 59.88 | | 100 | 57.75 | 58.16 |
| | 1000 | 133.62 | 86.69 | | 1000 | 128.3 | 106.9 |
| 26 | 1 | 12.11 | 9.62 | 37 | 1 | 11.21 | 8.41 |
| | 10 | 13.86 | 13.21 | | 10 | 11.50 | 09.07 |
| | 100 | 34.91 | 41.23 | | 100 | 17.57 | 17.67 |
| | 1000 | 64.45 | 76.21 | | 1000 | 45.57 | 60.45 |
| 27 | 1 | 13.24 | 10.10 | 38 | 1 | 10.05 | 11.78 |
| | 10 | 14.43 | 16.40 | | 10 | 16.41 | 13.56 |
| | 100 | 45.17 | 29.05 | | 100 | 25.26 | 24.99 |
| | 1000 | 76.38 | 106.77 | | 1000 | 76.16 | 82.16 |
| 28 | 1 | 16.19 | 10.19 | 39 | 1 | 10.61 | 11.53 |
| | 10 | 24.99 | 26.35 | | 10 | 11.83 | 11.16 |
| | 100 | 40.42 | 61.70 | | 100 | 13.51 | 15.76 |
| | 1000 | 54.48 | 58.94 | | 1000 | 29.46 | 40.41 |
| 29 | 1 | 17.55 | 10.56 | 40 | 1 | 10.65 | 10.06 |
| | 10 | 21.21 | 12.10 | | 10 | 9.07 | 9.01 |
| | 100 | 29.93 | 33.52 | | 100 | 10.07 | 10.90 |
| | 1000 | 64.22 | 68.55 | | 1000 | 16.69 | 36.47 |
| 31 | 1 | 13.73 | 11.80 | 41 | 1 | 12.56 | 6.51 |
| | 10 | 17.22 | 14.35 | | 10 | 12.79 | 06.74 |
| | 100 | 38.33 | 41.47 | | 100 | 17.70 | 14.03 |
| | 1000 | 39.25 | 50.32 | | 1000 | 46.10 | 58.08 |
| 32 | 1 | 20.40 | 11.78 | 42 | 1 | 17.72 | 7.30 |
| | 10 | 16.80 | 12.90 | | 10 | 9.90 | 04.48 |
| | 100 | 22.80 | 14.48 | | 100 | 20.41 | 15.00 |
| | 1000 | 69.80 | 64.95 | | 1000 | 91.42 | 60.84 |
| 33 | 1 | 18.71 | 14.04 | | | | |
| | 10 | 32.25 | 30.10 | | | | |
| | 100 | 52.13 | 72.54 | | | | |
| | 1000 | 74.03 | 62.11 | | | | |

* Fold Induction w.r.t T3(100 nm)

The data above clearly indicates that several of the novel compounds of the present invention are selective to TR-beta receptor and therefore have potential therapeutically beneficial properties.

### In-Vivo Studies:

Cholesterol lowering effect of T3 and selected compounds disclosed in the present invention on cholesterol lowering and change in heart rate in cholesterol-fed rats (treated for 7 days) was determined according to the general protocol described in PNAS, vol. 100 (17) 10067-10072 and Endocrinology 145(4):1656-1661 Many of the compounds were found to be reducing cholesterol and having very little effect on the heart rate. Therefore, these compounds have the potential to be further developed as selective TR-beta agonists for the treatment of human & other animals in need of such treatment.

| Ex. | Dose | % Change in | % Change in |
|---|---|---|---|
| No | (Oral) | Total Cholesterol | Heart Rate |
| T3 | 13 μg/kg | −46% | 14% |
| 3 | 0.5 mg/kg | −68% | 4.2% |
| 10 | 0.2 mg/kg | −45% | 9.5% |
| 14 | 0.1 mg/kg | −44% | −2% |

The novel compounds of the present invention may be formulated into suitable pharmaceutically acceptable compositions by combining with suitable excipients by techniques and processes and concentrations as are well known.

The compounds of formula (I) or pharmaceutical compositions containing them are useful as Thyroid hormone receptor ligands suitable for humans and other warm blooded animals, and may be administered either by oral, topical or parenteral administration for the treatment of various disease conditions associated with dyslipidemia, obesity etc.

The pharmaceutical composition is provided by employing conventional techniques. Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

We claim:

1. Accordingly, the present invention relates to compounds of the general formula (I), 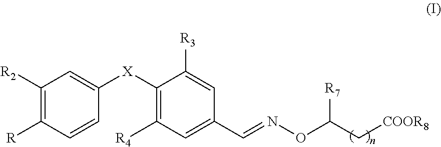
wherein R═OR₁, NHR₁, wherein R₁ is selected from H, or the groups selected from linear or branched (C₁-C₆)alkyl, (C₃-C₇)cycloalkyl, acyl, aryl, aralkyl groups, each of these groups being further substituted with suitable substituents; R₂ represents hydrogen, hydroxyl, halo, or the groups selected from (C₁-C₆)alkyl, (C₃-C₇)cycloalkyl, aryl, heteroaryl, acyl, oxo, aryloxy, aralkyl, aralkoxy, carboxylic acid and its derivatives such as esters and amides, sulfenyl derivatives, sulfonyl derivatives, sulfonic acid and its derivatives, each of these groups being further substituted with suitable substituents; or the groups —CONR₅R₆ , —SO₂NR₅R₆, wherein R₅ & R₆ are same or different and are independently selected from H, or the groups selected from linear or branched (C₁-C₆)alkyl, (C₃-C₇)cycloalkyl, bicycloalkyl, aryl or the groups, each of these groups being further substituted with suitable substituents, or R₅ & R₆ together with the nitrogen atom to which they are attached, form a five to eight membered cyclic ring which optionally contain one or more heteroatoms selected from N, S, O; R₃, R₄ are independently selected from H, halogen, (C₁-C₆)alkyl groups; X is selected from O, —CH₂—, CO; R₇ is selected from H, optionally substituted groups selected from linear or branched (C₁-C₆)alkyl, (C₃-C₇)cycloalkyl, acyl, aryl, aralkyl, heteroaryl groups and ‘n’ represents the integers 0-2; R₈ is selected from H, or the groups selected from linear or branched (C₁-C₆)alkyl groups, which may be further substituted with suitable substituents.

2. The compound as claimed in claim 1 wherein R₂ is selected linear or branched (C₁-C₆)alkyl, phenyl, benzoyl benzyl, carboxamide and sulfonamide groups, each of these groups being further substituted with suitable substituents.

3. The compound as claimed in claim 1 wherein R₈ represents an (C₁-C₆)alkyl group.

4. The compound of claim 1 wherein the aryl group is selected from phenyl, naphthyl, tetrahydronaphthyl, indane, biphenyl groups.

5. The compound of claim 1 wherein the heteroaryl group is selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthaiazynil, naphthylidinyl, purinyl, carbazolyl, phenothiazinyl, phenoxazinyl, benzoxazolyl, benzothiazolyl group.

6. The compounds as claimed in claim 1 wherein the substituents on alkyl, aryl, aralkyl, aryloxy, aralkoxy, heteroaryl or cycloalkyl groups are selected from hydroxyl, halo, cyano, optionally substituted groups selected from (C₁-C₆)alkyl, haloalkyl, alkoxy, oxo, aryl, aryloxy, aralkyl, acyl, alkylthio, thioalkyl groups.

7. The compounds as claimed in claim 1 wherein the substitutions on R₂ when present, is selected from halogen, hydroxy, amino, alkyl, haloalkyl, alkoxy groups.

8. The compounds as claimed in any preceding claims selected from
2-(((3,5-dichloro-4-(4-hydroxy-3-isopropyl phenoxy)benzylidene)amino)oxy)acetic acid;
2-(((4-(3-(sec-butyl)-4-hydroxyphenoxy)-3,5-dichlorobenzylidene)amino)oxy)acetic acid;
2-(((3,5-dibromo-4-(4-hydroxy-3-isopropylphenoxy)benzylidene)amino)oxy)acetic acid;
2-(((3,5-dichloro-4-((6-hydroxy-[1,1′-biphenyl]-3-yl)oxy)benzylidene)amino)oxy)acetic acid;
2-(((4-(3-benzyl-4-hydroxyphenoxy)-3,5-dichlorobenzylidene)amino)oxy)acetic acid;
2-(((3,5-dibromo-4-(3-(sec-butyl)-4-hydroxyphenoxy)benzylidene)amino)oxy)acetic acid;
2-(((3,5-dibromo-4-((6-hydroxy-[1,1′-biphenyl]-3-yl)oxy)benzylidene)amino)oxy)acetic acid;
2-(((3,5-dichloro-4-(4-hydroxy-3-isopropylphenoxy)benzylidene)amino)oxy)propanoic acid;
2-(((4-(3-benzyl-4-hydroxyphenoxy)-3,5-dibromobenzylidene)amino)oxy)acetic acid;
2-(((3,5-dibromo-4-(4-hydroxy-3-isopropylphenoxy)benzylidene)amino)oxy)propanoic acid;
2-(((3,5-dibromo-4-((6-hydroxy-[1,1′-biphenyl]-3-yl)oxy)benzylidene)amino)oxy)propanoic acid;
2-(((3,5-dibromo-4-(4-hydroxy-3-isopropylphenoxy)benzylidene)amino)oxy)-2-methylpropanoic acid;
2-(((3,5-dibromo-4-(4-hydroxy-3-isopropylphenoxy)benzylidene)amino)oxy)butanoic acid;
2-(((3,5-dibromo-4-(3 -(sec-butyl)-4-hydroxyphenoxy)benzylidene)amino)oxy)propanoic acid;
2-(((3,5-dibromo-4-(3-(sec-butyl)-4-hydroxyphenoxy)benzylidene)amino)oxy)-2-methyl prop anoic acid;
2-(((4-(3-benzyl-4-hydroxyphenoxy)-3,5-dibromobenzylidene)amino)oxy)propanoic acid;
2-(((4-(3-benzyl-4-hydroxyphenoxy)-3,5-dibromobenzylidene)amino)oxy)butanoic acid;
2-(((3,5-dibromo-4-(3-(sec-butyl)-4-hydroxyphenoxy)benzylidene)amino)oxy)-2-phenylacetic acid;
2-(((3,5-dibromo-4-(3-(sec-butyl)-4-hydroxyphenoxy)benzylidene)amino)oxy)butanoic acid;
2-(((3,5-dibromo-4-((6-hydroxy-[1,1′-biphenyl]-3-yl)oxy)benzylidene)amino)oxy)-2-methyl propanoic acid;
2-(((3,5-dibromo-44(6-hydroxy-[1,1′-biphenyl]-3-yl)oxy)benzylidene)amino)oxy)butanoic acid;
2-(((4-(3-benzyl-4-hydroxyphenoxy)-3,5-dibromobenzylidene)amino)oxy)-2-methyl propanoic acid;
2-(((4-(3-(sec-butyl)-4-hydroxyphenoxy)-3,5-dichlorobenzylidene)amino)oxy)propanoic acid;
2-(((3,5-dibromo-4-(3-(tert-butyl)-4-hydroxyphenoxy)benzylidene)amino)oxy)propanoic acid;
2-(((3,5-dibromo-4-(3-ethyl-4-hydroxyphenoxy)benzylidene)amino)oxy)propanoic acid;
2-(((3,5-dibromo-4-(3-(4-chlorobenzoyl)-4-hydroxy phenoxy)benzylidene)amino)oxy)propan oic acid;
2-(((3,5-dibromo-4-(3-((4-chlorophenyl)(hydroxy)methyl)-4-hydroxy phenoxy)benzylidene)amino)oxy)propanoic acid;
2-(((3,5-dibromo-4-(3-(4-chlorobenzoyl)-4-hydroxyphenoxy)benzylidene)amino)oxy)acetic acid;
2-(((3,5-dibromo-4-(3-((4-chlorophenyl)(hydroxy)methyl)-4-hydroxyphenoxy)benzylidene)amino)oxy)acetic acid;
2-(((3,5-dibromo-4-(3-(3-chlorobenzoyl)-4-hydroxy phenoxy)benzylidene)amino)oxy)propanoic acid;
2-(((3,5-dibromo-4-(3-(4-bromobenzoyl)-4-hydroxyphenoxy)benzylidene)amino)oxy)acetic acid;
2-(((3,5-dibromo-4-(3-((4-bromophenyl)(hydroxy)methyl)-4-hydroxyphenoxy)benzylidene)amino)oxy)acetic acid;
2-(((3,5-dibromo-4-(3-(3-chlorobenzoyl)-4-hydroxyphenoxy)benzylidene)amino)oxy)acetic acid;
2-(((3,5-dibromo-4-(3-((3-chlorophenyl)(hydroxy)methyl)-4-hydroxyphenoxy)benzylidene)amino)oxy)propanoic acid;
2-(((3,5-dibromo-4-(3-((3-chlorophenyl)(hydroxy)methyl)-4-hydroxyphenoxy)benzylidene)amino)oxy)acetic acid;
2-(((3,5-dibromo-4-(4-hydroxy-3-(piperidin-1-ylsulfonyl)phenoxy)benzylidene)amino)oxy)propanoic acid;
2-(((3,5-dibromo-4-(4-hydroxy-3-(N-isopropylsulfamoyl)phenoxy)benzylidene)amino)oxy)propanoic acid;
2-(((3,5-dibromo-4-(3-(N,N-diethylsulfamoyl)-4-hydroxyphenoxy)benzylidene)amino)oxy)propanoic acid;
2-(((3,5-dibromo-4-(3-(N-cyclohexylsulfamoyl)-4-hydroxyphenoxy)benzylidene)amino)oxy)propanoic acid;
2-(((4-(3-(N-((1R,2R,4S)-bicyclo[2.2.1]heptan-2-yl)sulfamoyl)-4-hydroxyphenoxy)-3,5-dibromobenzylidene)amino)oxy)propanoic acid;
2-(((3,5-dibromo-4-(4-hydroxy-3-(pyrrolidin-1-ylsulfonyl)phenoxy)benzylidene)amino)oxy)acetic acid;
2-(((3,5-dibromo-4-(4-hydroxy-3-tosylphenoxy)benzylidene)amino)oxy)propanoic acid.

9. A pharmaceutical composition which comprises compounds of formula (I), as claimed in any preceding claims and a pharmaceutically acceptable carrier, diluent or excipients.

10. A method of preventing or treating diseases caused by dyslipidemia or obesity comprising administering an effective, non-toxic amount of compound of formula (I) or suitable pharmaceutical composition as defined in any preceding claims to a patient in need thereof.

11. A medicine for treating/reducing dyslipidemia or obesity which comprises administering a compound of formula (I), as defined in any preceding claims and a pharmaceutically acceptable carrier, diluent or excipients to a patient in need thereof.

12. Use of compounds of formula (I), their pharmaceutical compositions and medicines containing them as defined in any previous claims as a medicament suitable for the treatment of diseases mentioned in any of the aforesaid claims.

13. Intermediates selected from
Ethyl 2-(((3,5-dichloro-4-(3-isopropyl-4-methoxyphenoxy)benzylidene)amino)oxy)acetate;
Ethyl 2-(((4-(3-(sec-butyl)-4-methoxyphenoxy)-3,5-dichlorobenzylidene)amino)oxy)acetate;
Ethyl 2-(((3,5-dibromo-4-(3-isopropyl-4-methoxyphenoxy)benzylidene)amino)oxy)acetate;
Ethyl 2-(((3,5-dichloro-4-((6-methoxy-[1,1′-biphenyl]-3-yl)oxy)benzylidene)amino)oxy)acetate;
Ethyl 2-(((4-(3-benzyl-4-methoxyphenoxy)-3,5-dichlorobenzylidene)amino)oxy)acetate;
Ethyl 2-(((3,5-dibromo-4-(3-(sec-butyl)-4-methoxyphenoxy)benzylidene)amino)oxy)acetate;
Ethyl 2-(((3,5-dibromo-4-((6-methoxy-[1,1′-biphenyl]-3-yl)oxy)benzylidene)amino)oxy)acetate;
Ethyl 2-(((3,5-dichloro-4-(3-isopropyl-4-methoxyphenoxy)benzylidene)amino)oxy)propanoate;
Ethyl 2-(((4-(3-benzyl-4-methoxyphenoxy)-3,5-dibromobenzylidene)amino)oxy)acetate;
Ethyl 2-(((3,5-dibromo-4-(3-isopropyl-4-methoxyphenoxy)benzylidene)amino)oxy)propanoate;
Ethyl 2-(((3,5-dibromo-4-((6-methoxy-[1,1′-biphenyl]-3-yl)oxy)benzylidene)amino)oxy)propanoate;
Ethyl 2-(((3,5-dibromo-4-(3-isopropyl-4-methoxyphenoxy)benzylidene)amino)oxy)-2-methyl propanoate;
Ethyl 2-(((3,5-dibromo-4-(3-isopropyl-4-methoxyphenoxy)benzylidene)amino)oxy)butanoate;
Ethyl 2-(((3,5-dibromo-4-(3-(sec-butyl)-4-methoxyphenoxy)benzylidene)amino)oxy)propanoate;
Ethyl 2-(((3,5-dibromo-4-(3-(sec-butyl)-4-methoxyphenoxy)benzylidene)amino)oxy)-2-methylpropanoate;
Ethyl 2-(((4-(3-benzyl-4-methoxyphenoxy)-3,5-dibromobenzylidene)amino)oxy)propanoate;
Ethyl 2-(((4-(3-benzyl-4-methoxyphenoxy)-3,5-dibromobenzylidene)amino)oxy)butanoate;
Ethyl 2-(((3,5-dibromo-4-(3-(sec-butyl)-4-methoxyphenoxy)benzylidene)amino)oxy)-2-phenyl acetate;
Ethyl 2-(((3,5-dibromo-4-(3-(sec-butyl)-4-ethoxy phenoxy)benzylidene)amino)oxy)butanoate;
Ethyl 2-(((3,5 -dibromo-4-((6-methoxy-[1,1′-biphenyl]-3-yl)oxy)benzylidene)amino)oxy)-2-methyl propanoate;
Ethyl 2-(((3,5-dibromo-4-((6-methoxy-[1,1′-biphenyl]-3-yl)oxy)benzylidene)amino)oxy)butanoate;
Ethyl 2-(((4-(3-benzyl-4-methoxyphenoxy)-3,5-dibromobenzylidene)amino)oxy)-2-methyl propanoate;
Ethyl 2-(((4-(3 -(sec-butyl)-4-methoxyphenoxy)-3,5-dichlorobenzylidene)amino)oxy)propanoate;
Ethyl 2-(((3,5-dibromo-4-(3-(tert-butyl)-4-methoxyphenoxy)benzylidene)amino)oxy)propanoate;
Ethyl 2-(((3,5-dibromo-4-(3-ethyl-4-methoxyphenoxy)benzylidene)amino)oxy)propanoate;
Ethyl 2-(((3,5-dibromo-4-(3-(4-chlorobenzoyl)-4-methoxyphenoxy)benzylidene)amino)oxy)propanoate;
Ethyl 2-(((3,5-dibromo-4-(3-(4-chlorobenzoyl)-4-hydroxyplienoxy)benzylidene)amino)oxy)propanoate;
Ethyl 2-(((3,5-dibromo-4-(3-(4-chlorobenzoyl)-4-hydroxyphenoxy)benzylidene)amino)oxy)acetate;
Ethyl 2-(((3,5-dibromo-4-(3-(3-chlorobenzoyl)-4-hydroxyphenoxy)benzylidene)amino)oxy)propanoate;
Ethyl 2-(((3,5-dibromo-4-(3-(4-bromobenzoyl)-4-hydroxyphenoxy)benzylidene)amino)oxy)acetate;
Ethyl 2-(((3,5-dibromo-4-(3-(3-chlorobenzoyl)-4-hydroxyphenoxy)benzylidene)amino)oxy)acetate;
Ethyl 2-(((3,5-dibromo-4-(4-methoxy-3-(piperidin-1-ylsulfonyl)phenoxy)benzylidene)amino)oxy)propanoate;
Ethyl 2-(((3,5-dibromo-4-(3-(N-isopropylsulfamoyl)-4-methoxyphenoxy)benzylidene)amino)oxy)propanoate;
Ethyl 2-(((3,5-dibromo-4-(3-(N,N-diethylsulfamoyl)-4-methoxyphenoxy)benzylidene)amino)oxy)propanoate;
Ethyl 2-(((3,5-dibromo-4-(3-(N-cyclohexylsulfamoyl)-4-methoxyphenoxy)benzylidene)amino)oxy)propanoate;
Ethyl 2-(((4-(3-(N-((1R,2R,4S)-bicyclo[2.2.1]heptan-2-yl)sulfamoyl)-4-methoxyphenoxy)-3,5-dibromobenzylidene)amino)oxy)propanoate;
Ethyl 2-(((3,5-dibromo-4-(4-methoxy-3-(pyrrolidin-1-ylsulfonyl)phenoxy)benzylidene)amino)oxy)acetate;
Ethyl 2-(((3,5-dibromo-4-(4-methoxy-3-(pyrrolidin-1-ylsulfonyl)phenoxy)benzylidene)amino)oxy)propanoate;

14. The compounds as claimed in claim 13 suitable as intermediates for the preparation of compounds of formula (I).
